(19) United States
(12) Patent Application Publication
(10) Pub. No.: US 2003/0113917 A1
(43) Pub. Date: Jun. 19, 2003

(54) Methods for delivering nucleic acid molecules into cells and assessment thereof

(75) Inventors: Gary de Jong, North Vancouver (CA); Sandra Louise Vanderbyl, Burnaby (CA); Jan Drayer, San Francisco, CA (US); Paul Peter Tak, Leiden (NL)

Correspondence Address: Stephanie L. Seidman, HELLER EHRMAN WHITE & McAULIFFE LLP, 7th Floor, 4350 La Jolla Village Drive, San Diego, CA, 92122-1246, US

(73) Assignee: Chromos Molecular Systems, Inc.

(21) Appl. No.: 10/235,119

(22) Filed: Sep. 3, 2002

Related U.S. Application Data
(63) Continuation of application No. 10/086,745, filed on Feb. 28, 2002.
(63) Continuation-in-part of application No. 09/815,981, filed on Mar. 22, 2001.

(51) Int. Cl. C12N 5/08; C12N 5/06; C12N 15/85

(52) U.S. Cl. 435/455; 435/366; 435/325

(57) ABSTRACT

Methods for delivering nucleic acid molecules into cells and methods for measuring nucleic acid delivery into cells and the expression of the nucleic acids are provided. The methods are designed for introduction of large nucleic acid molecules, including artificial chromosomes, into cells, and are practiced in vitro and in vivo.

### RELATED APPLICATIONS

This application is a continuation of U.S. application Ser. No. 10/086,745, filed Feb. 28, 2002, to de Jong et al. entitled “METHODS FOR DELIVERING NUCLEIC ACID MOLECULES INTO CELLS AND ASSESSMENT THEREOF.” This application is a continuation-in-part of U.S. application Ser. No. 09/815,981 filed Mar. 22, 2001, to de Jong et al. entitled “METHODS FOR DELIVERING NUCLEIC ACID MOLECULES INTO CELLS AND ASSESSMENT THEREOF.” This application is related to U.S. application Ser. No. 09/815,979, filed Mar. 22, 2001, to de Jong et al., entitled “METHODS FOR DELIVERING NUCLEIC ACID MOLECULES INTO CELLS AND ASSESSMENT THEREOF.”The subject matter of each of these applications is incorporated in its entirety by reference thereto.

### FIELD OF THE INVENTION

The present invention relates to methods of delivering nucleic acid molecules into cells and methods for measuring nucleic acid delivery into cells and the expression of the nucleic acids therein.

### BACKGROUND OF THE INVENTION

A number of methods of delivering nucleic acid molecules, particularly plasmid DNA and other small fragments of nucleic acid, into cells have been developed. These methods are not ideal for delivery of larger nucleic acid molecules. Thus, there is a need for methods of delivering nucleic acid molecules of increasing size and complexity, such as artificial chromosomes, into cells. Methods are required for use with in vitro and in vivo procedures such as gene therapy and for production of transgenic animals and plants. Furthermore, there is a need for the ability to rapidly and simply determine and assess the efficiency of delivery of DNA into cells.

Therefore it is an object herein to provide methods for delivering nucleic acid molecules, particularly larger molecules, including artificial chromosomes, into cells. Methods for assessing delivery are also provided.

### SUMMARY OF THE INVENTION

Methods for delivery of large nucleic acid molecules into cells are provided. The methods, which can be used to deliver nucleic acid molecules of any size, are suitable for delivery of larger nucleic acid molecules, such as natural and artificial chromosomes and fragments thereof, into cells. The methods are designed for in vitro, ex vivo and in vivo delivery of nucleic acid molecules for applications, including, but not limited to, delivery of nucleic acid molecules to cells for cell-based protein production, transgenic protein production and gene therapy. Methods of protein production in cells and in transgenic animals and plants, methods of introducing nucleic acid into cells to produce transgenic animals and plants, and methods for ex vivo and in vivo gene therapy are also provided.

Methods provided herein are designed for delivering a large nucleic acid molecule into a cell, but also can be used to deliver smaller molecules. The methods include the steps of exposing the nucleic acid molecule to a first delivery agent, typically an agent that increases contact between the nucleic acid molecule and the cell; and exposing the cell to a second delivery agent, which is generally different from the first agent, and is particularly an agent, such as energy, that enhances permeability of the cell. Selected delivery agents and combinations thereof are those that result in delivery of the nucleic acid into the cell to a greater extent than in absence of the agent or in the presence of one of the agents alone. In all of these methods, if the permeability enhancing agent is energy, such as electroporation or sonoporation, the cell is contacted therewith in the absence of the nucleic acid molecule.

Also provided are methods in which the cells are contacted with a lipid agent, particularly a dendrimer, such as SAINT-2™ (1-methyl-4-(1-octadec-9-enyl-nonadec-10-enylenyl) pyridinium chloride, also designated 1-methyl-4-(19-cis,cis-hepatritiaconta-9,28-dienyl) pyridinium chloride), simultaneously with or sequentially with application of energy. The nucleic acid, which is optionally, although preferably not treated with a delivery agent, is contacted with the so-treated cell.

The selected delivery methods vary depending on the target cells (cells into which nucleic acid is delivered), the nucleic acid molecules, and the type(s) of delivery agent(s) selected. Exemplary methods for delivery of large nucleic acid molecules into cells provided herein include, but are not limited to, methods involving any of the following:

mixing the nucleic acid molecule with a delivery agent, such as a cationic lipid that neutralizes the charge of the nucleic acid, and contacting the cell with the mixture of nucleic acid and delivery agent;

contacting a cell with the nucleic acid molecule, and then contacting the cell with a delivery agent or contacting a cell with a delivery agent then contacting the cell with the nucleic acid molecule;

contacting a cell in the absence of the nucleic acid molecule with a delivery agent, applying ultrasound or electrical energy to the cell contacted with the delivery agent, and contacting the cell with the nucleic acid molecule upon the conclusion of the application of the energy;

applying ultrasound or electrical energy to a cell, and contacting the cell, upon conclusion of the application of the energy, with a mixture of the nucleic acid molecule and a delivery agent;

applying ultrasound or electrical energy to a cell, contacting the cell with a delivery agent upon conclusion of the application of the energy and contacting the cell previously contacted with the delivery agent with the nucleic acid molecule;

applying ultrasound or electrical energy to a cell and contacting the cell with the nucleic acid molecule upon conclusion of the application of the energy;

contacting a cell in the absence of the nucleic acid molecule with a delivery agent, applying ultrasound or electrical energy to the cell contacted with the delivery agent, and contacting the cell with a mixture of the nucleic acid and a delivery agent upon the conclusion of the application of the energy.

Although combinations of the above methods can be used, it has been found that any application of energy to the cells must be done prior to introduction of the nucleic acid molecule.

The methods provided herein are intended for delivery of large nucleic acid molecules into cells in a variety of environments for a variety of purposes. For example, nucleic acid molecules greater than about 0.5, 0.6. 0.7, 0.8, 0.9, 1, 5, 10, 30, 50 and 100 megabase pairs can be delivered into cells using the methods provided herein. The methods can be used to deliver the large nucleic acid molecules into cells in vitro or in vivo.

In in vivo applications of the delivery methods, such as in in vivo gene therapy, large nucleic acid molecules can be delivered to cells directly in an animal subject. Such animals include, but are not limited to mammals. For example, the animal subject can be a human or other primate, rodent, rabbit, dog, horse or monkey. Reagents can be administered locally or systemically (e.g., in the bloodstream) in the subject. For example, local administration of the nucleic acids, and/or delivery agents, can be into areas such as joints, the skin, tissues, tumors and organs. For systemic administration, the nucleic acid molecules can be targeted to cells or tissues of interest.

The delivery methods provided herein also can be used to deliver large nucleic acid molecules to a target cell in vitro, which is then introduced into an animal subject, in particular human subjects, such as can be done, for example, in a method of ex vivo gene therapy. Thus, also provided herein are methods of in vivo and ex vivo gene therapy using the methods for delivering large nucleic acid molecules into cells as provided herein.

In particular embodiments of the methods in which a delivery agent is used, the delivery agent is a cationic compound. Cationic compounds include, but are not limited to, a cationic lipid, a cationic polymer, a mixture of cationic lipids, a mixture of cationic polymers, a mixture of a cationic lipid and a cationic polymer and a mixture of a cationic lipid and a neutral lipid, polycationic lipids, non-liposomal forming lipids, activated dendrimers, ethanolic cationic lipids, cationic amphiphiles and pyridinium chloride surfactants.

Included among the nucleic acid molecules that can be delivered into cells using the methods provided herein are artificial chromosomes, satellite DNA-based artificial chromosomes (SATACs, herein referred to as ACes) and natural chromosomes or fragments of any of these chromosomes.

The ultrasound energy can be applied as one continuous pulse or two or more intermittent pulses. The intermittent pulses of the ultrasound energy can be applied for substantially the same length of time, at substantially the same energy level or can vary in energy level, the length of time applied, or energy level and the length of time applied. Ultrasound energy ranges and number of pulses can vary, from methods provided herein, according to the instrument selected and can be empirically determined. Typically, ultrasound will be applied for about 30 seconds to about 5 minutes. The power used is a function of the sonorporator used.

The effects of the ultrasound energy can be enhanced by contacting a cell (in vitro) or administering to a subject (in vivo) a cavitation compound prior to the application of ultrasound energy. Thus, the provided methods can include the use of such cavitation compounds.

When electric fields are employed in the methods provided herein, they are preferably applied to the cells in suspension for about 20 to 50 msec, but the timing and voltage is a function of the instrument used and the particular parameters. The electrical energy can be applied as one to five intermittent pulses. As noted, electrical field ranges and number of pulses can vary according to instrument specification and can be determined empirically.

Methods are provided for generating transgenic animals, particularly non-human transgenic animals, by delivering large nucleic acid molecules into animal cells, in particular non-human animal cells, using delivery methods provided herein, and exposing the animal cells into which the large nucleic acid molecules are delivered to conditions whereby a transgenic animal develops therefrom.

The methods for delivering large nucleic acid molecules into cells provided herein also can be used in methods of generating transplantable organs and tissues. Exemplary cells for use in methods of generating transgenic animals, particularly non-human transgenic animals, or transplantable organs include, but are not limited to, an embryonic stem cell, a nuclear transfer donor cell, a stem cell and a cell that is capable of the generation of a specific organ. The methods for delivering nucleic acid molecules into cells provided herein also can be used in methods of generating cellular protein production cell lines.

Further provided are methods for monitoring delivery of nucleic acids into a cell. These methods permit the rapid and accurate measurement of nucleic acid transfer into cells, thus allowing for screening and optimizing the use of various delivery agents and protocols for delivery of any nucleic acid into any cell type, in vitro, ex vivo or in vivo. Further provided are methods to monitor delivery and expression of nucleic acids in a cell.

In embodiments of the methods for monitoring delivery of nucleic acids into a cell, labeled nucleic acid molecules, such as DNA, are delivered into the cell using the delivery agent(s) as described herein, or using any delivery method known to those of skill in the art. A detection method, such as flow cytometry, is then used to determine the number of cells containing the label as an indication of the ability of the delivery method to facilitate or effect delivery of the nucleic acid molecules. Other detection methods that can be used in place of or in addition to flow cytometry include, but are not limited to, fluorimetry, cell imaging, fluorescence spectroscopy and other such methods known to those of skill in the art for such detection and, as needed or desired, for quantitation.

In an exemplary embodiment of the methods for monitoring and quantifying delivery of nucleic acid molecules, such as DNA, into cells, the nucleic acid molecule is an artificial chromosome labeled with a nucleoside or ribonucleoside analog, particularly a thymidine analog, such as iododeoxyuridine (IdU or IdUrd) and bromodeoxyuridine (BrdU), and the delivery agent is a cationic compound, which is used alone or in combination with energy.

Because of the ease with which numbers of events are collected, the monitoring methods provided herein, particularly those based on flow cytometry techniques, provide a method for collection of nucleic acid molecule delivery data that is statistically superior to previous methods of evaluating nucleic acid molecules transfer. The positive values are instrument derived and therefore are not susceptible to judgement errors.

The monitoring methods provided herein permit the rapid, simple and accurate detection of delivery of small numbers of nucleic acid molecules into cells. Such small numbers can be sufficient for purposes of transgenesis, gene therapy, cellular protein production and other goals of gene transfer. The monitoring methods also make it possible to rapidly quantify differences in delivery efficiencies of differing delivery methods and thus facilitate the development and optimization of methods for the delivery of nucleic acid molecules, such as DNA, into cells.

These methods also can be used to optimize transfection efficiencies into cells for which no delivery protocol has been established or which are not easily transfected. These methods also permit rapid screening of delivery protocols and agents for their ability to enhance or permit delivery of nucleic acid molecules, such as DNA, of any size into a cell.

Methods are also provided that combine methods of monitoring nucleic acid molecule delivery with methods for monitoring expression of nucleic acid molecules. It is possible not only to assess the efficiency of delivery of nucleic acid molecules to cells, but also to monitor the subsequent expression of the delivered nucleic acid molecules in the same cell population. Thus, these methods also provide a method for the mapping of biological events between nucleic acid molecule delivery and early gene expression, using marker genes, such as, but are not limited to, fluorescent proteins, such as red, green or blue fluorescent proteins. In a particular embodiment of these combined methods, delivery and expression of nucleic acid molecules, such as delivery of a chromosome and expression of genes encoded thereon, are monitored by IdU labeling of a nucleic acid molecule that contains sequences encoding a green fluorescent protein.

In particular embodiments, the methods of monitoring delivery and expression of a nucleic acid molecule include the steps of: introducing labelled nucleic acid molecules that encode a reporter gene into cells; detecting labelled cells as an indication of delivery of the nucleic acid into a cell; and measuring the product of the reporter gene as an indication of DNA expression in the cell, whereby delivery and expression of nucleic acid molecules in the cell is detected or determined. The labelled cells can be detected, for example, by flow cytometry, fluorimetry, cell imaging or fluorescence spectroscopy. The label, for example, can be iododeoxyuridine (IdU or IdUrd) or bromodeoxyuridine (BrdU), the reporter gene, for example, can be one that encodes fluorescent protein, enzyme, such as a luciferase, or antibody. The delivered nucleic acid molecules include, but are not limited to, RNA, including ribozymes, DNA, including naked DNA and chromosomes, plasmids, chromosome fragments, typically containing at least one gene or at least 1 Kb, naked DNA, or natural chromosomes. The method is exemplified herein by determining delivery and expression of artificial chromosome expression systems (ACes). Any types of cells, eukaryotic and prokaryotic, including cell lines, primary cells, primary cell lines, plant cells, and animal cells, including stem cells, embryonic cells, and other cells into which delivery of a nucleic acid molecule can occur, are contemplated and can be used in the methods provided herein.

### DETAILED DESCRIPTION OF THE INVENTION

A. Definitions

Unless defined otherwise, all technical and scientific terms used herein have the same meaning as is commonly understood by one of skill in the art to which this invention belongs. All patents, patent applications and publications referred to herein are incorporated by reference.

As used herein, “nucleic acid” refers to a polynucleotide containing at least two covalently linked nucleotide or nucleotide analog subunits. A nucleic acid can be a deoxyribonucleic acid (DNA), a ribonucleic acid (RNA), or an analog of DNA or RNA. Nucleotide analogs are commercially available and methods of preparing polynucleotides containing such nucleotide analogs are known (Lin et al. (1994) Nucl. Acids Res. 22:5220-5234; Jellinek et al. (1995) Biochemistry 34:11363-11372; Pagratis et al. (1997) Nature Biotechnol. 15:68-73). The nucleic acid can be single-stranded, double-stranded, or a mixture thereof. For purposes herein, unless specified otherwise, the nucleic acid is double-stranded, or it is apparent from the context.

The term “nucleic acid” refers to single-stranded and/or double-stranded polynucleotides, such as deoxyribonucleic acid (DNA) and ribonucleic acid (RNA), as well as analogs or derivatives of either RNA or DNA. Also included in the term “nucleic acid” are analogs of nucleic acids such as peptide nucleic acid (PNA), phosphorothioate DNA, and other such analogs and derivatives.

As used herein, DNA is meant to include all types and sizes of DNA molecules including cDNA, plasmids and DNA including modified nucleotides and nucleotide analogs.

As used herein, nucleotides include nucleoside mono-, di-, and triphosphates. Nucleotides also include modified nucleotides, such as, but are not limited to, phosphorothioate nucleotides and deazapurine nucleotides and other nucleotide analogs.

As used herein, the term “large nucleic acid molecules” or “large nucleic acids” refers to a nucleic acid molecule of at least about 0.5 megabase pairs (Mbase) in size, greater than 0.5 Mbase, including nucleic acid molecules at least about 0.6. 0.7, 0.8, 0.9, 1, 5, 10, 30, 50 and 100, 200, 300, 500 Mbase in size. Large nucleic acid molecules typically can be on the order of about 10 to about 450 or more Mbase, and can be of various sizes, such as, for example, from about 250 to about 400 Mbase, about 150 to about 200 Mbase, about 90 to about 120 Mbase, about 60 to about 100 Mbase and about 15 to 50 Mbase.

Examples of large nucleic acid molecules include, but are not limited to, natural chromosomes and fragments thereof, especially mammalian chromosomes and fragments thereof which retain a centromere and telomeres, artificial chromosome expression systems (ACes; also called satellite DNA-based artificial chromosomes (SATACs); see U.S. Pat. Nos. 6,025,155 and 6,077,697), mammalian artificial chromosomes (MACs), plant artificial chromosomes, insect artificial chromosomes, avian artificial chromosomes and minichromosomes (see, e.g., U.S. Pat. Nos. 5,712,134, 5,891,691 and 5,288,625). The large nucleic acid molecules can include a single copy of a desired nucleic acid fragment encoding a particular nucleotide sequence, such as a gene of interest, or can carry multiple copies thereof or multiple genes or different heterologous sequences of nucleotides. For example, ACes can carry 40 or even more copies of a gene of interest. Large nucleic acid molecules can be associated with proteins, for example chromosomal proteins, that typically function to regulate gene expression and/or participate in determining overall structure.

As used herein, an artificial chromosome is a nucleic acid molecule that can stably replicate and segregate alongside endogenous chromosomes in a cell. It has the capacity to act as a gene delivery vehicle by accommodating and expressing foreign genes contained therein. A mammalian artificial chromosome (MAC) refers to chromosomes that have an active mammalian centromere(s). Plant artificial chromosomes, insect artificial chromosomes and avian artificial chromosomes refer to chromosomes that include plant, insect and avian centromeres, respectively. A human artificial chromosome (HAC,) refers to chromosomes that include human centromeres. For exemplary artificial chromosomes, see, e.g., U.S. Pat. Nos. 6,025,155; 6,077,697; 5,288,625; 5,712,134; 5,695,967; 5,869,294; 5,891,691 and 5,721,118 and published International PCT application Nos, WO 97/40183 and WO 98/08964.

As used herein, the term “satellite DNA-based artificial chromosome (SATAC)” is interchangeable with the term “artificial chromosome expression system (ACes)”. These artificial chromosomes are substantially all neutral non-coding sequences (heterochromatin) except for foreign heterologous, typically gene-encoding nucleic acid, that is interspersed within the heterochromatin for the expression therein (see U.S. Pat. Nos. 6,025,155 and 6,077,697 and International PCT application No. WO 97/40183). Foreign genes contained in these artificial chromosome expression systems can include, but are not limited to, nucleic acid that encodes traceable marker proteins (reporter genes), such as fluorescent proteins, such as green, blue or red fluorescent proteins (GFP, BFP and RFP, respectively), other reporter genes, such as β-galactosidase and proteins that confer drug resistance, such as a gene encoding hygromycin-resistance. Other examples of heterologous DNA include, but are not limited to, DNA that encodes therapeutically effective substances, such as anti-cancer agents, enzymes and hormones, and DNA that encodes other types of proteins, such as antibodies.

As used herein, the terms “heterologous” and “foreign” with reference to nucleic acids, such as DNA and RNA, are used interchangeably and refer to nucleic acid that does not occur naturally as part of a genome or cell in which it is present or which is found in a location(s) and/or in amounts in a genome or cell that differ from the location(s) and/or amounts in which it occurs in nature. It can be nucleic acid that is not endogenous to the cell and has been exogenously introduced into the cell. Examples of heterologous DNA include, but are not limited to, DNA that encodes a gene product or gene product(s) of interest introduced into cells, for example, for purposes of gene therapy, production of transgenic animals or for production of an encoded protein. Other examples of heterologous DNA include, but are not limited to, DNA that encodes traceable marker proteins, such as a protein that confers drug resistance, DNA that encodes therapeutically effective substances, such as anti-cancer agents, enzymes and hormones, and DNA that encodes other types of proteins, such as antibodies.

As used herein, “delivery,” which is used interchangeably with “transfection,” refers to the process by which exogenous nucleic acid molecules are transferred into a cell such that they are located inside the cell. Delivery of nucleic acids is a distinct process from expression of nucleic acids.

As used herein, “expression” refers to the process by which nucleic acid is translated into peptides or is transcribed into RNA, which, for example, can be translated into peptides, polypeptides or proteins. If the nucleic acid is derived from genomic DNA, expression can, if an appropriate eukaryotic host cell or organism is selected, include splicing of the mRNA. For heterologous nucleic acid to be expressed in a host cell, it must initially be delivered into the cell and then, once in the cell, ultimately reside in the nucleus.

As used herein, cell recovery refers to a “total cell yield” after a specified time frame, which for purposes herein is twenty-four hours, and when used with reference to calculation of the clonal fraction

As used herein, cell recovery time refers to a time frame in order for a cell to equilibrate to new conditions.

As used herein, cell survival refers to cell viability after a cytotoxic event, such as a delivery procedure.

As used herein, control plating efficiency (CPE) refers to the fraction of untreated cells, under standard optimal growth conditions for the particular cells, that survive a plating procedure. Plating efficiency refers to the fraction of treated cells that survive a plating procedure.

As used herein, clonal fraction is a measurement of cell recovery after delivery of exogenous nucleic acids into cells and the plating efficiency of the cells.

As used herein, transfer efficiency is the percentage of the total number of cells to which nucleic acids are delivered that contain delivered nucleic acid.

As used herein, transfection efficiency is the percentage of the total number of cells to which nucleic acids including a selectable marker are delivered that survive selection.

As used herein, index of potential transfection efficiency means the theoretical maximum transfection efficiency for a particular cell type under particular conditions, for example particular concentrations or amounts of particular delivery agents.

As used herein, the term “cell” is meant to include cells of all types, of eukaryotes and prokaryotes, including animals and plants.

As used herein, “delivery agent” refers to compositions, conditions or physical treatments to which cells and/or nucleic acids can be exposed in the process of transferring nucleic acids to cells in order to facilitate nucleic acid delivery into cells. Delivery agents include compositions, conditions and physical treatments that enhance contact of nucleic acids with cells and/or increase the permeability of cells to nucleic acids. In all instances, nucleic acids are not directly treated with energy, such as sonoporation.

As used herein, cationic compounds are compounds that have polar groups that are positively charged at or around physiological pH. These compounds facilitate delivery of nucleic acid molecules into cells; it is thought this is achieved by virtue of their ability to neutralize the electrical charge of nucleic acids. Exemplary cationic compounds include, but are not limited to, cationic lipids or cationic polymers or mixtures thereof, with or without neutral lipids, polycationic lipids, non-liposomal forming lipids, ethanolic cationic lipids and cationic amphiphiles. Contemplated cationic compounds also include activated dendrimers, which are spherical cationic polyamidoamine polymers with a defined spherical architecture of charged amino groups which branch from a central core and which can interact with the negatively charged phosphate groups of nucleic acids (e.g., starburst dendrimers).

Cationic compounds for use as delivery agents also include mixtures of cationic compounds that include peptides and protein fragments. The additional components can be non-covalently or covalently bound to the cationic compound or otherwise associated with the cationic compound.

As used herein, ultrasound energy is meant to include sound waves (for external application) and lithotripter-generated shock waves (for internal application).

As used herein, electrical energy is meant to include the application of electric fields to cells so as to open pores in membranes for the delivery of molecules into the cell, e.g., electroporation techniques.

As used herein, cavitation compound is meant to include contrast agents that are typically used with ultrasound imaging devices and includes gas encapsulated and nongaseous agents. These cavitation compounds enhance the efficiency of energy delivery of acoustic or shock waves.

As used herein, “pharmaceutically acceptable” as used herein refers to compounds, compositions and dosage forms that are suitable for administration to the subject without causing excessive toxicity, irritation, allergic response or other undesirable complication.

As used herein, embryonic stem cells are primitive, immature cells that are precursors to stem cells.

As used herein, stem cells are primitive, immature cells that are precursors to mature, tissue specific cells.

As used herein, nuclear transfer donor cells are cells that are the source of nuclei, which are transferred to enucleated oocytes during the process of nuclear transfer.

As used herein, the term “subject” refers to animals, plants, insects, and birds into which the large DNA molecules can be introduced. Included are higher organisms, such as mammals and birds, including humans, primates, rodents, cattle, pigs, rabbits, goats, sheep, mice, rats, guinea pigs, cats, dogs, horses, chicken and others.

As used herein, “administering to a subject” is a procedure by which one or more delivery agents and/or large nucleic acid molecules, together or separately, are introduced into or applied onto a subject such that target cells which are present in the subject are eventually contacted with the agent and/or the large nucleic acid molecules.

As used herein, “applying to a subject” is a procedure by which target cells present in the subject are eventually contacted with energy such as ultrasound or electrical energy. Application is by any process by which energy can be applied.

As used herein, gene therapy involves the transfer or insertion of nucleic acid molecules, and, in particular, large nucleic acid molecules, into certain cells, which are also referred to as target cells, to produce specific gene products that are involved in correcting or modulating diseases or disorders. The nucleic acid is introduced into the selected target cells in a manner such that the nucleic acid is expressed and a product encoded thereby is produced. Alternatively, the nucleic acid can in some manner mediate expression of DNA that encodes a therapeutic product. This product can be a therapeutic compound, which is produced in therapeutically effective amounts or at a therapeutically useful time. It can also encode a product, such as a peptide or RNA, that in some manner mediates, directly or indirectly, expression of a therapeutic product. Expression of the nucleic acid by the target cells within an organism afflicted with a disease or disorder thereby provides a way to modulate the disease or disorder. The nucleic acid encoding the therapeutic product can be modified prior to introduction into the cells of the afflicted host in order to enhance or otherwise alter the product or expression thereof.

For use in gene therapy, cells can be transfected in vitro, followed by introduction of the transfected cells into the body of a subject. This is often referred to as ex vivo gene therapy. Alternatively, the cells can be transfected directly in vivo within the body of a subject.

As used herein, flow cytometry refers to processes that use a laser based instrument capable of analyzing and sorting out cells and/or chromosomes based on size and fluorescence.

As used herein, a reporter gene includes any gene that expresses a detectable gene product, which can be RNA or protein. Preferred reporter genes are those that are readily detectable. Examples of reporter genes include, but are not limited to nucleic acid encoding a fluorescent protein, CAT (chloramphenicol acetyl transferase) (Alton and Vapnek (1979) Nature 282:864-869), luciferase, and other enzyme detection systems, such as beta-galactosidase; firefly luciferase (deWet et al. (1987), Mol. Cell. Biol. 7: 725-737); bacterial luciferase (Engebrecht and Silverman (1984), Proc. Natl. Acad. Sci. U.S.A. 1: 4154-4158; Baldwin et al. (1984), Biochemistry 23: 3663-3667); and alkaline phosphatase (Toh et al. (1989) Eur. J. Biochem. 182: 231-238, Hall et al. (1983) J. Mol. Appl. Gen. 2:101).

As used herein, a reporter gene construct is a DNA molecule that includes a reporter gene operatively linked to a transcriptional control sequence. The transcriptional control sequences include the promoter and other optional regulatory regions, such as enhancer sequences, that modulate the activity of the promoter, or control sequences that modulate the activity or efficiency of the RNA polymerase that recognizes the promoter, or control sequences are recognized by effector molecules, including those that are specifically induced by interaction of an extracellular signal with a cell surface protein. For example, modulation of the activity of the promoter can be effected by altering the RNA polymerase binding to the promoter region, or, alternatively, by interfering with initiation of transcription or elongation of the mRNA. Such sequences are herein collectively referred to as transcriptional control elements or sequences. In addition, the construct can include sequences of nucleotides that alter translation of the resulting mRNA, thereby altering the amount of reporter gene product.

As used herein, promoter refers to the region of DNA that is upstream with respect to the direction of transcription of the transcription initiation site. It includes the RNA polymerase binding and transcription imitation sites and any other regions, including, but not limited to repressor or activator protein binding sites, calcium or cAMP responsive sites, and any such sequences of nucleotides known to those of skill in the art to alter the amount of transcription from the promoter, either directly or indirectly.

As used herein, a promoter that is regulated or mediated by the activity of a cell surface protein is a promoter whose activity changes when a cell is exposed to a particular extracellular signal by virtue of the presence of cell surface proteins whose activities are affected by the extracellular protein.

B. Methods for the Delivery of DNA into Cells

A variety of methods for delivering nucleic acids, particularly large nucleic acid molecules, such as artificial chromosomes, including ACes (formerly designated SATACs), are provided. The methods generally involve exposing the nucleic acid molecule to an agent that increases contact between the nucleic acid molecule and the cell, and exposing the cell to a permeability enhancing agent. Each of the methods provided herein requires the use of one or both of these agents, which are applied in different orders, with the caveat that agents, such as energy, which increase the permeability of a cell, must be applied before contacting the cell with a nucleic acid.

In methods provided herein, large nucleic acid molecules are delivered using agents, including, but not limited to, delivery agents that enhance contact between the nucleic acid molecules and the cells and/or agents and treatments that increase cell permeability. Generally the nucleic acid molecules are delivered using agents that enhance contact between the nucleic acid and cells by neutralizing the charge of the nucleic acid molecules, and also by using energy to increase permeability of the cells. The agents can be used individually and in various combinations and orders of application, with the caveat that energy, such as sonoporation and electroporation cannot be applied to cells after the nucleic acid molecule is added thereto.

The method selected for delivering particular nucleic acid molecules, such as DNA, to targeted cells can depend on the particular nucleic acid molecule being transferred and the particular recipient cell. Preferred methods for particular nucleic acid molecules, such as DNA, and recipient cells are those that result in the greatest amount of nucleic acid molecules, such as DNA, transferred into the cell nucleus with an acceptable degree of cell survival. Suitable methods for delivery of particular pairings of nucleic acid molecules, such as DNA, and recipient cells can be determined using methods of monitoring nucleic acid molecules, such as DNA, delivery and methods of screening agents and conditions as provided herein or can be determined empirically using methods known to those of skill in the art.

The method selected requires consideration of a number of parameters, which are discussed in detail below. A method for detection of delivered nucleic acid is provided. This method, which can be used for assessing delivery of any nucleic acid molecule, can be used as a rapid screening tool to optimize chromosome transfer conditions.

In particular, delivery methods can first be assessed for the ability to transfer nucleic acid molecules, such as DNA, into cells and to identify methods that provide a sufficient number of viable cells that express the transferred nucleic acid molecules, such as DNA. Once such methods are identified, they can be optimized using the delivery monitoring methods provided herein and then assessed for the ability to provide for expression of the transferred nucleic acid molecules.

Delivery Agents

Delivery agents include compositions, conditions and physical treatments that enhance contact of nucleic acid molecules, such as DNA, with cells and/or increase the permeability of cells to nucleic acid molecules, such as DNA. Such agents include, but are not limited to, cationic compounds, peptides, proteins, energy, for example ultrasound energy and electric fields, and cavitation compounds.

Delivery agents for use in the methods provided herein include compositions, conditions or physical treatments to which cells and/or nucleic acid molecules, such as DNA, can be exposed in the process of transferring nucleic acid molecules, such as DNA, to cells in order to facilitate nucleic acid molecules, such as DNA, delivery into cells. For example, compounds and chemical compositions, including, but are not limited to, calcium phosphate, DMSO, glycerol, chloroquine, sodium butyrate, polybrene and DEAE-dextran, peptides, proteins, temperature, light, pH, radiation and pressure are all possible delivery agents.

Cationic Compounds

Cationic compounds for use in the methods provided herein are available commercially or can be synthesized by those of skill in the art. Any cationic compound can be used for delivery of nucleic acid molecules, such as DNA, into a particular cell type using the provided methods. One of skill in the art by using the provided screening procedures can readily determine which of the cationic compounds are best suited for delivery of specific nucleic acid molecules, such as DNA, into a specific target cell type.

(a) Cationic Lipids

Cationic lipid reagents can be classified into two general categories based on the number of positive charges in the lipid headgroup; either a single positive charge or multiple positive charges, usually up to 5. Cationic lipids are often mixed with neutral lipids prior to use as delivery agents. Neutral lipids include, but are not limited to, lecithins; phosphatidylethanolamine; phosphatidylethanolamines, such as DOPE (dioleoylphosphatidylethanolamine), DPPE (dipalmitoylphosphatidylethanolamine), dipalmiteoylphosphatidylethanolamine, POPE (palmitoyloleoylphosphatidylethanolamine) and distearoylphosphatidylethanolamine; phosphatidylcholine; phosphatidylcholines, such as DOPC (dioleoylphosphidylcholine), DPPC (dipalmitoylphosphatidylcholine), POPC (palmitoyloleoylphosphatidylcholine) and distearoylphosphatidylcholine; fatty acid esters; glycerol esters; sphingolipids; cardiolipin; cerebrosides; and ceramides; and mixtures thereof. Neutral lipids also include cholesterol and other 3βOH-sterols.

Other lipids contemplated herein, include: phosphatidylglycerol; phosphatidylglycerols, such as DOPG (dioleoylphosphatidylglycerol), DPPG (dipalmitoylphosphatidylglycerol), and distearoylphosphatidylglycerol; phosphatidylserine; phosphatidylserines, such as dioleoyl- or dipalmitoylphosphatidylserine and diphosphatidylglycerols.

Examples of cationic lipid compounds include, but are not limited to: Lipofectin (Life Technologies, Inc., Burlington, Ont.)(1:1 (w/w) formulation of the cationic lipid N-[1-(2,3-dioleyloxy)propyl]-N,N,N-trimethylammonium chloride (DOTMA) and dioleoylphosphatidylethanolamine (DOPE)); LipofectAMINE (Life Technologies, Burlington, Ont., see U.S. Pat. No. 5,334,761) (3:1 (w/w) formulation of polycationic lipid 2,3-dioleyloxy-N-[2(spermine-carboxamido)ethyl]-N,N-dimethyl-1-propanaminiumtrifluoroacetate (DOSPA) and dioleoylphosphatidylethanolamine (DOPE)), LipofectAMINE PLUS (Life Technologies, Burlington, Ont. see U.S. Pat. Nos. 5,334,761 and 5,736,392; see, also U.S. Pat. No. 6,051,429) (LipofectAmine and Plus reagent); LipofectAMINE 2000 (Life Technologies, Burlington, Ont.; see also International PCT application No. WO 00/27795) (Cationic lipid); Effectene (Qiagen, Inc., Mississauga, Ontario) (Non liposomal lipid formulation), Metafectene (Biontex, Munich, Germany) (Polycationic lipid); Eu-fectins (Promega Biosciences, Inc., San Luis Obispo, Calif.) (ethanolic cationic lipids numbers 1 through 12:C₅₂H₁₀₆N₆O₄.4CF₃CO₂H, C₈₈H₁₇₈N₈O₄S₂.4CF₃CO₂H, C₄₀H₈₄NO₃P.CF₃CO₂H, C₅₀H₁₀₃N₇O₃.4CF₃CO₂H, C₅₅H₁₁₆N₈O₂.6CF₃CO₂H, C₄₉H₁₀₂N₆O₃.4CF₃CO₂H, C₄₄H₈₉N₅O₃.2CF₃CO₂H, C₁₀₀H₂₀₆N₁₂O₄S₂.8CF₃CO₂H, C₁₆₂H₃₃₀N₂₂O₉.13CF₃CO₂H, C₄₃H₈₈N₄O₂.2CF₃CO₂H, C₄₃H₈₈N₄O₃.2CF₃CO₂H, C₄₁H₇₈NO₈P); Cytofectene (Bio-Rad, Hercules, Calif.) (mixture of a cationic lipid and a neutral lipid); GenePORTER (Gene Therapy Systems Inc., San Diego, Calif.) (formulation of a neutral lipid (DOPE) and a cationic lipid) and FuGENE 6 (Roche Molecular Biochemicals, Indianapolis, Ind.) (Multi-component lipid based non-liposomal reagent).

(b) Non-Lipid Cationic Compounds

Non-lipid cationic reagents include, but are not limited to SUPERFECT™ (Qiagen, Inc., Mississauga, ON) (Activated dendrimer (cationic polymer:charged amino groups)) and CLONfectin™ (Cationic amphiphile N-t-butyl-N′-tetradecyl-3-tetradecyl-aminopropionamidine) (Clontech, Palo Alto, Calif.).

Pyridinium amphiphiles are double-chained pyridinium compounds, which are essentially nontoxic toward cells and exhibit little cellular preference for the ability to transfect cells. Examples of a pyridinium amphiphiles are the pyridinium chloride surfactants such as SAINT-2 (1-methyl-4-(1-octadec-9-enyl-nonadec-10-enylenyl) pyridinium chloride) (see, e.g., van der Woude et al. (1997) Proc. Natl. Acad. Sci. U.S.A. 94:1160). The pyridinium chloride surfactants are typically mixed with neutral helper lipid compounds, such as dioleoylphosphatidylethanolamine (DOPE), in a 1:1 molar ratio. Other Saint derivatives of different chain lengths, state of saturation and head groups can be made by those of skill in the art and are within the scope of the present methods.

Energy

Delivery agents also include treatment or exposure of the cell and/or nucleic acid molecules, but generally the cells, to sources of energy, such as sound and electrical energy.

Ultrasound

For in vitro and in vivo transfection, the ultrasound source should be capable of providing frequency and energy outputs suitable for promoting transfection. Preferably, the output device can generate ultrasound energy in the frequency range of 20 kHz to about 1 MHz. The power of the ultrasound energy is preferably in the range from about 0.05 w/cm² to 2 w/cm², more preferably from about 0.1 w/cm² to about 1 w/cm². The ultrasound can be administered in one continuous pulse or can be administered as two or more intermittent pulses, which can be the same or can vary in time and intensity.

Ultrasound energy can be applied to the body locally or ultrasound-based extracorporeal shock wave lithotripsy can be used for “in-depth” application. The ultrasound energy can be applied to the body of a subject using various ultrasound devices. In general, ultrasound can be administered by direct contact using standard or specially made ultrasound imaging probes or ultrasound needles with or without the use of other medical devices, such as scopes, catheters and surgical tools, or through ultrasound baths with the tissue or organ partially or completely surrounded by a fluid medium. The source of ultrasound can be external to the subject's body, such as an ultrasound probe applied to the subject's skin which projects the ultrasound into the subject's body, or internal, such as a catheter having an ultrasound transducer which is placed inside the subject's body. Suitable ultrasound systems are known (see, e g., International PCT application No. WO 99/21584 and U.S. Pat. No. 5,676,151).

When the cationic compound and nucleic acid molecules, such as DNA, are administered systemically, the ultrasound can be applied to one or several organs or tissues simultaneously to promote nucleic acid molecule delivery to multiple areas of the subject's body. Alternatively, the ultrasound can be applied selectively to specific areas or tissues to promote selective uptake of the nucleic acid molecules, such as DNA.

The transfection efficiency of the ultrasound also can be enhanced by using contrast reagents, which serve as artificial cavitation nuclei, such as Albunex (Molecular Biosystems, San Diego, Calif.), Imagent (Alliance Pharmaceutical, San Diego, Calif.), Levovist-SHU (Schering AG, Berlin, Germany), Definity (E. I. du Pont de Nemour, Wilmington, Del.), STUC (Washington University, St Louis, Mo.) and the introduction of gaseous microbubbles. A contrast reagent can be introduced locally, such as a joint; introduced systematically, with the enhancement of cavitation efficiency by focusing lithotripter shock waves at a defined area; or by targeting a contrast reagent to a particular site and then enhancing cavitation efficiency by focusing lithotripter shock waves.

Electroporation

Electroporation temporarily opens up pores in a cell's outer membrane by use of pulsed rotating electric fields. Methods and apparatus used for electroporation in vitro and in vivo are well known (see, e.g., U.S. Pat. Nos. 6,027,488, 5,993,434, 5,944,710, 5,507,724, 5,501,662, 5,389,069, 5,318,515). Standard protocols can be employed.

C. Target Cells and Delivery Thereto

The methods provided herein can be used in the delivery of nucleic acids into any cells, including, but not limited to, any eukaryotic and prokaryotic cells. Examples of cells that can be used in the methods include, but are not limited to, cell lines, primary cells, primary cell lines, plant cells and animal cells, including stem cells and embryonic cells. For example, fibroblasts, including lung and skin fibroblasts, fibroblast-like cells, synoviocytes, fibroblast-like synoviocytes, stem cells, including embryonic and adult stem cells, such as mesenchymal stem cells, myoblasts, lymphoblasts, carcinoma and hepatoma cells are among the many cells into which nucleic acids, and in particular large nucleic acids and artificial chromosomes, can be delivered and monitored using the methods provided herein. Particular cells include mammalian cells, for example, A9 cells (mouse fibroblasts, HPRT⁻; ATCC Accession no. CCL-1.4), CHO-S cells and DG44 cells (Chinese hamster ovary cells), V79 cells (Chinese hamster lung fibroblasts; ATCC Accession no. CCL-39), LMTK⁻ cells (mouse fibroblasts; ATCC Accession No. CCL-1.3), skin fibroblasts, L8 cells (rat myoblasts; ATCC Accession No. CRL-1769), CCD1043 SK cells (human fibroblasts; ATCC Accession No. CRL-2056), adult-derived mesenchymal stem cells (e.g., derived from human bone marrow; Cambrex Biosciences, East Rutherford, N.J.), synoviocytes (rat and human), Detroit 551 cells (human embryonic skin fibroblasts; ATCC Accession No. CCL-110), NSO (murine myeloma, ECACC Accession No. 85110503), 293 cells (human embryonic kidney cells transformed by type 5 (Ad 5) DNA (ATCC Accession No. CRL-1573), P46-Fl (bovine lymphocyte-like cell line), DT40 (chicken lymphoblasts), EJ30 cells (human bladder carcinoma), HepG2 cells (human hepatoma) and murine and bovine embryos.

In particular embodiments, the methods of delivery of nucleic acids into cells provided herein can be used in delivering nucleic acids into cells in order to treat a disease or disorder, e.g., in gene therapy applications. In gene therapy applications, the nucleic acid to be delivered into a cell can encode a therapeutic molecule, e.g., a protein. In many instances, successful gene therapy applications are complicated by a requirement that large nucleic acids be delivered into cells. It also can be desired to provide multiple copies of nucleic acid encoding a therapeutic molecule. Compounding the difficulties in gene therapy methods is the challenge that cells preferred for use in gene therapy applications are often not readily transfectable. The methods provided herein are particularly well suited for delivery of large nucleic acids, which can be in the form of artificial chromosomes or fragments thereof, into cells as can be used in therapeutic applications.

In Vitro Delivery

Cationic compounds and nucleic acid molecules, such as DNA, can be added to cells in vitro either separately or mixed together and with or without the application of ultrasound or electrical energy. In particular embodiments, if energy is applied, it is applied prior to contacting the cells with the nucleic acid molecule.

In general, nucleic acid molecules, such as DNA, mixed with cationic lipids/compounds can be added to a cell as described in the EXAMPLES. Parameters important for optimization of the delivery of nucleic acid molecules, such as DNA, into target cells will be apparent to those of skill in this art. These parameters include, for example, the cationic compound, cationic compound concentration, the nucleic acid molecules, such as DNA, the concentration of nucleic acid molecules, the cell growth medium, the cell culture conditions, the length of time cells are exposed to the cationic compound, the toxicity of the cationic compound to the target cell type, and the amount and time of use of ultrasound or electroporation among other parameters. It can be necessary to optimize these parameters for different nucleic acid molecules, such as DNA, and target cell types. Such optimization is routine employing the guidance provided herein. In addition, the rapid screening method can provide direction as to what parameters can need to be adjusted to optimize delivery (see EXAMPLES). Alteration of culture conditions, time, reagent concentrations and other parameters, for use with different combinations of cationic compounds and target cell types and to optimize delivery, can be empirically determined. If ultrasound energy is required to be used to enhance transfection efficiency, it can be applied as described below and in the EXAMPLES. Electroporation can be performed as described below or by any suitable protocol known to those of skill in this art.

The contacting of cells with cationic compounds and nucleic acid molecules, such as DNA, in separate and distinct steps can be generally carried out as described in the EXAMPLES. Those of skill in the art can readily vary the order of the application of the components to the target cell based on the disclosure herein.

Ex Vivo Gene Therapy

Delivery of nucleic acid molecules, such as DNA, is carried out as described above in in vitro delivery. After selection has been completed, cells harboring the nucleic acid molecules, such as DNA, are introduced into the subject target by a variety of means, including injection, such as subcutaneous, intramuscular, intraperitoneal, intravascular and intralymphatic and intra-articular injection. The cells can be administered with or without the aid of medical devices such as arthroscopes, other scopes or various types of catheters.

In Vivo Gene Therapy

In one method for delivering of the nucleic acid molecules, such as DNA, to target cells in the body of a subject in vivo, the cationic compound is first delivered to the target area (e.g., tissue, organ, tumor or joint). After waiting a suitable amount of time, the target area is then subjected to ultrasound frequency at a suitable energy level for a suitable time, which will be dependent on the equipment, tissue type and depth of the target area in the body. Alternatively, electrical energy is delivered to the target area. The nucleic acid molecule, such as DNA, is then delivered to the same area. Optionally, this procedure can be repeated so that the nucleic acid molecules, such as DNA, can be delivered via multiple injections over time or multiple administrations in different areas at the same time.

The cationic compound mixed together with the nucleic acid molecules, such as DNA, can be delivered to the target area. The target area can then be subjected to ultrasound frequency at a suitable energy level for a suitable time. Depending on the nucleic acid molecules, such as DNA, the in vivo location, the cationic compound used and other variables, it can not be necessary to use ultrasound or electroporation to achieve suitable transfer efficiency to cells at the target area. Prior to the application of ultrasound, contrast reagents can be delivered to the target area to enhance transfer of the nucleic acid molecules, such as DNA.

The nucleic acid molecules, such as DNA, can be delivered to organs or tissues of the body such as skin, muscle, stomach, intestine, lung, bladder, ovary, uterus, liver, kidney, pancreas, brain, heart, spleen, prostate and joints (for example the knee, elbow, shoulder, wrist, hip, finger, ankle and others). Molecules can be delivered to, for example, primary cells and cell lines, such as fibroblast, muscle, stomach, intestine, lung, bladder, ovary, uterus, liver, kidney, pancreas, brain, heart, spleen, prostate to mimic in vivo systems.

The cationic compounds and the nucleic acid molecules, such as DNA, separately or together can be delivered to the target area of the body by a variety of means, including injection (for example, subcutaneous, intramuscular, intraperitoneal, intravascular, intra-articular and intralymphatic injection), instillation, cannulation, slow infusion, topical application and any other mode of administration. They can be administered by any suitable mode, including systemically (for example by intravenous injection), locally, such as by delivery to a specific target area (tissue or area), using, for example, a catheter or by direct injection. They can be administered with or without the aid of medical devices such as arthroscopes, other scopes or various types of catheters.

The cationic compounds can be administered also by coating a medical device, for example, a catheter, such as an angioplasty balloon catheter, with a cationic compound formulation. Coating can be achieved, for example, by dipping the medical device into a cationic lipid formulation or a mixture of a cationic compound formulation and a suitable solvent, for example, an aqueous-buffer, an aqueous solvent, ethanol, methylene chloride, chloroform and other suitable solvent. An amount of the formulation will naturally adhere to the surface of the device, which is subsequently administered to a subject, as appropriate. Alternatively, a lyophilized mixture of a cationic lipid formulation can be specifically bound to the surface of the device. Such binding techniques are known (see, e.g., Ishihara et al. (1993) Journal of Biomedical Materials Research 27:1309-1314).

The cationic compounds and nucleic acid molecules, such as DNA, can be formulated in pharmaceutically acceptable carriers, such as saline or other pharmaceutically acceptable solutions, for delivery in vivo. The nucleic acid molecules, such as DNA, and cationic compounds, regardless of the route of administration, are formulated into pharmaceutically acceptable dosage forms by standard methods known to those of skill in the art.

For gene therapy, the dosage level of the nucleic acid molecules, such as DNA, can be varied to achieve optimal therapeutic response for a particular subject. This depends on a variety of factors including mode of administration, activity of the nucleic acid molecules, such as DNA, characteristics of the protein produced, the transfection efficiency of the target cells (their ability to take up the nucleic acid molecules, such as DNA), the route of administration, the location of the target cells and other factors

The dosage to be administered and the particular mode of administration will vary depending upon such factors as the age, weight and the particular animal and region thereof to be treated, the particular nucleic acid molecule and cationic compound used, the therapeutic or diagnostic use contemplated, and the form of the formulation, for example, suspension, emulsion, or liposomal, as will be readily apparent to those skilled in the art. Typically, dosage is administered at lower levels and increased until the desirable therapeutic effect is achieved. The amount of cationic compound that is administered can vary and generally depends upon the amount of nucleic acid molecules, such as DNA, being administered. For example, the weight ratio of cationic compound to nucleic acid molecules is preferably from about 1:1 to about 15:1, with a weight ratio of about 5:1 to about 1:1 being more preferred. Generally, the amount of cationic compound which is administered will vary from between about 0.1 milligram (mg) to about 1 gram (g). By way of general guidance, typically for a bodyweight of 70 kg and a composition with about 1 to 10 million chromosomes per ml, single dose ranging from 1 to 20 ml is administered as a single or repeated dose.

For localized treatment of diseases, such administration to affected joints in rheumatoid arthritis, psoriasis and diabetes should be possible, as well as injection into muscle for treatment of diseases, such as hemophilia or other genetic diseases. For other than local treatment a targeted delivery step is needed.

Cells for effecting treatment of disease are also provided. For example, compositions are provided that contain cells selected for therapeutic treatment of a joint or rheumatoid arthritis, wherein the cells contain a large heterologous nucleic acid. For example, the cells of such a composition can contain an artificial chromosome. In a particular embodiment of such compositions, the cells can contain an ACes.

Gene Therapy in Connective Tissue and Rheumatic Diseases

Rheumatoid arthritis (RA) is a chronic inflammatory disease characterized by joint inflammation and progressive cartilage and bone destruction. Treatment of RA is problematic with current strategies since relatively high systemic doses are necessary to achieve therapeutic levels of anti-rheumatic drugs in the joints. In addition, the available treatments are associated with significant untoward side effects. Gene therapy is thus a more efficient system for delivery of therapeutic molecules to the site of inflammation in the treatment of connective tissue diseases, rheumatic diseases and chronic erosive joint diseases such as RA, osteoarthritis, ankylosing spondylitis and juvenile chronic arthritis.

In a diarthrodial movable joint, smooth articulation is ensured by the macromolecular structure of the articular cartilage which covers the ends of the bones. The cavity or joint space that occurs at the location of adjacent bones is lined by a tissue referred to as the synovium. The synovium contains macrophage-like type A cells (presumably derived from macrophage/monocyte precursors and exhibiting phagocytic activity) and fibroblast-like type B cells (more fibroblast in appearance and associated with production of hyaluronic acid and other components of the joint fluid). Underlying the synovium is a sparsely cellular subsynovium which can be fibrous, adipose or areolar in nature. Fibroblast-like synoviocytes are distinguishable from normal fibroblast cells in the subintimal synovium by differential gene expression patterns. FLS have been shown to express high levels of uridine diphosphoglucose dehydrogenase (UDPGD), high levels of vascular cell adhesion molecule-1 (VCAM-1), intercellular adhesion molecule-1 (ICAM-1) as well as CD44 (hyaluronic acid receptor), fibronectin receptor and β3 integrins. Sublining fibroblasts or fibroblasts from other sources do not express these markers or express them at lower levels [see, e.g., Edwards (1995) Ann. Rheum. Dis. 54:395-397; Firestein (1996) Arthritis Rheum. 39:1781-1790; Edwards (2000) Arthritis Res. 2:344-347].

Disease progression in RA involves the thickening of the synovial lining due to the proliferation of fibroblast-like synoviocytes (FLS) and infiltration by inflammatory cells (e.g., lymphocytes, macrophages and mast cells). The normal biology of synoviocytes is also altered in the pathological process of RA, including invasion and destruction of articular cartilage and bone. In addition to the production of elastase and collagenase, synoviocytes mediate the pathophysiological process of RA by expression of cell surface proteins involved in the recruitment and activation of lymphocytes and macrophages within the synovium. Proliferation of synovial cells leads to a pannus tissue that invades and overgrows cartilage, leading to bone destruction and destruction of joint structure and function. Proinflammatory cytokines, for example, tumor necrosis factor-α (TNF-α) and interleukin-1 (IL-1) play key roles in inflammation and joint damage associated with RA. Pathological effects caused by these cytokines include leukocytic infiltration leading to synovial hyperplasia, cell activation, cartilage breakdown and inhibition of cartilage matrix synthesis.

Nucleic acid transfer to rheumatoid synovial tissue can result in the production of mediators that inhibit inflammation or hyperplasia or provide toxic substances that specifically destroy the diseased synovium. Retroviral delivery of nucleic acid encoding interleukin-1 receptor antagonist (IL1-RA) ex vivo and transduction of synoviocytes has been used in gene therapy of RA in humans to inhibit inflammation [see, e.g., Evans (1996) Human Gen. Ther. 7:1261-1280 and Del Vecchio et al. (2001) Arthritis Res. 3:259-263]. Adenoviral vectors have been proposed for delivery of nucleic acid encoding an IL-1 receptor antagonist to synoviocytes in in vivo transduction methods [see, e.g., U.S. Pat. No. 5,747,072 and PCT Application Publication No. WO 00/52186].

Artificial chromosomes provide advantages over virus-based systems for gene therapy. For example, artificial chromosome expression systems (ACes), and other artificial chromosomes as described in U.S. Pat. Nos. 6,025,155 and 6,077,697 and PCT Application No. WO97/40183, serve as non-integrating, non-viral vectors with a large capacity for delivering large nucleic acids and/or multiple copies of a particular nucleotide sequence into cells, such as synoviocytes, both in vitro and in vivo. Such artificial chromosome systems offer further advantages in that they allow stable and predictable expression of genes producing single or multiple proteins over long periods of time.

The methods provided herein can be used to introduce large nucleic acids, such as, for example, artificial chromosomes, into primary cells, such as synoviocytes (e.g., fibroblast-like synoviocytes) and skin fibroblasts, and skeletal muscle fibroblast cell lines. Thus, included among the methods provided herein is a method for introducing heterologous nucleic acid into a synoviocyte by introducing in a chromosome, such as, for example, an artificial chromosome, into the synoviocyte. In one such embodiment, the artificial chromosome is an ACes. The synoviocyte can be, for example, a fibroblast-like synoviocyte.

A particular method provided herein for introducing a large nucleic acid molecule into a synoviocyte includes steps of exposing the nucleic acid molecule to a delivery agent and contacting the synoviocyte with the nucleic acid molecule. In a particular embodiment of this method, the delivery agent is not energy. In one embodiment, the large nucleic acid molecule is a chromosome. For example, the nucleic acid can be an artificial chromosome, such as an ACes. In a particular embodiment, the synoviocyte is a fibroblast-like synoviocyte. Any delivery agents, such as described herein, can be used in such methods. For example, the delivery agent can be one that includes a cationic compound.

Also provided is a method for introducing a nucleic acid molecule into a synoviocyte that includes steps of exposing the nucleic acid molecule to a delivery agent, exposing the synoviocyte to a delivery agent and contacting the synoviocyte with the nucleic acid molecule, whereby the nucleic acid molecule is delivered into the synoviocyte, and wherein the steps are performed sequentially in any order or simultaneously. In some embodiments of the method, if the delivery agent is energy, it is not applied to the nucleic acid molecule and it is not applied to the synoviocyte after contacting the synoviocyte with the nucleic acid molecule. The nucleic acid can be any nucleic acid. In particular embodiments, the nucleic acid is a large nucleic acid, chromosome, artificial chromosome or ACes. In a further particular embodiment, the synoviocyte is a fibroblast-like synoviocyte. Delivery agents, such as described herein, can be used in such methods. For example, the delivery agent can be one that includes a cationic compound.

Another method for delivering a nucleic acid molecule into a synoviocyte provided herein includes steps of contacting the synoviocyte in the presence or absence of the nucleic acid molecule with a delivery agent, and applying ultrasound energy or electrical energy to the synoviocyte, wherein the contacting and applying are performed sequentially or simultaneously, and then contacting the synoviocyte with the nucleic acid molecule, whereby the nucleic acid molecule is delivered into the synoviocyte. The nucleic acid can be any nucleic acid. In particular embodiments, the nucleic acid is a large nucleic acid, chromosome, artificial chromosome or ACes. In a further particular embodiment, the synoviocyte is a fibroblast-like synoviocyte. Numerous delivery agents, including agents such as those described herein, can be used in such methods. For example, the delivery agent can be one that includes a cationic compound. In one embodiment, the energy is ultrasound.

Thus, provided herein are methods of delivering nucleic acids, in particular, large nucleic acids, such as chromosomes, including artificial chromosomes, e.g., ACes, into primary cells, including synoviocytes and fibroblasts. These methods can be used in vitro and in vivo.

Also provided herein is a synoviocyte comprising a large heterologous nucleic acid, a heterologous chromosome or portion thereof, or an artificial chromosome. In one embodiment, the artificial chromosome is an ACes. Such synoviocytes include fibroblast-like synoviocytes. The synoviocytes can be from any species, including, but not limited to mammalian species. For example, synoviocytes containing large nucleic acids, such as for example artificial chromosomes (e.g., ACes) include primate synoviocytes, as well as rodent, rabbit, monkey and human synoviocytes.

The ability to achieve delivery of large nucleic acids into such cells demonstrates the usefulness of the methods in gene therapy applications as well as in the testing in animal models of disease of possible therapeutic molecules for use in gene therapy methods. Thus, provided herein are methods of treating diseases or modulating disease processes which include steps of introducing a large nucleic acid molecule, chromosome or portion thereof, or artificial chromosome into a subject who has the disease.

A method for modulating a rheumatic disease process in a subject is provided herein. In one embodiment, the method includes steps of introducing a large nucleic acid into a site of inflammation in the subject, wherein the large nucleic acid contains nucleic acid that is or that encodes an agent that modulates a rheumatic disease process. Processes associated with rheumatic diseases are known in the art and are described herein. For example, one such process is an inflammatory process that includes processes of cell activation, infiltration, proliferation and recruitment. In a particular embodiment of this method, the disease is rheumatoid arthritis. The nucleic acid can be, for example, a chromosome or portion thereof or an artificial chromosome, e.g., an ACes. One possible site of inflammation is a joint.

Also provided is a method for treating a rheumatic disease in a subject in which a large nucleic acid is introduced into the subject, wherein the large nucleic acid contains nucleic acid that is or that encodes a therapeutic agent. In a particular embodiment of this method, the disease is rheumatoid arthritis. The nucleic acid can be, for example, a chromosome or portion thereof or an artificial chromosome, e.g., an ACes. In an embodiment of this method, the disease is rheumatoid arthritis.

In the methods for modulating a rheumatic disease process or treating a rheumatic disease, the method can be practiced in any format, including ex vivo and in vivo formats. Thus, for example, the nucleic acid can be introduced into a cell in vitro and then transferred into the subject. Alternatively, the nucleic acid can be introduced into a cell in vivo. In a particular embodiment, the nucleic acid is introduced into a synoviocyte, which can be, for example, a fibroblast-like synoviocyte. The nucleic acid that is introduced can comprise any nucleic acid that is or that encodes a molecule that has an anti-rheumatic effect in the subject. For example, the molecule can alter, counteract or diminish a process of the disease. The molecule can ameliorate symptoms of the disease. Molecules that provide anti-rheumatic effects in subjects with RA are known in the art [see, e.g., Vervoordeldonk and Tak (2001) Best Prac. Res. Clin. Rheumatol. 15:771-788 and WO 00/52186]. Such molecules include anti-inflammatory or immunomodulatory molecules. For example, interleukin-1 receptor antagonists, soluble interleukin-1 receptor, soluble tumor necrosis factor receptor, interferon-β, interleukin-4, interleukin-10, interleukin-13, transforming growth factor β, dominant negative IkappaB-kinase, FasL, Fas-associated death domain protein or CTLA-4 are among molecules that can have anti-rheumatic effects.

Also provided is a method of evaluating or testing a nucleic acid as a potential therapeutic agent in the treatment of a connective tissue or rheumatic disease by introducing into an animal model of a connective tissue or rheumatic disease a large nucleic acid molecule. The nucleic acid molecule can be one that includes nucleic acid that is or encodes a candidate therapeutic agent. The method can include a step of determining if the nucleic acid molecule has any effects, and in particular any anti-rheumatic effects, on the animal. For example, in determining if the nucleic acid molecule has any effects on the animal, it can be evaluated whether one or more conditions of the disease is effected, such as, for example, amelioration of or reduction in an adverse condition. In a particular embodiment, the disease is a rheumatic disease, such as, for example rheumatoid arthritis. The animal is any animal in which the disease can be modeled. For example, the animal can be a mammal. In particular embodiments the animal is a monkey, rodent, rabbit, dog, cat, horse, cow, pig or primate. The large nucleic acid can be, for example, a chromosome, or portion thereof, or an artificial chromosome, for example, an ACes. In particular embodiments, the nucleic acid molecule is in a synoviocyte, such as, for example, a fibroblast-like synoviocyte. In further embodiments, the nucleic acid is introduced into a joint of the animal. The nucleic acid molecule can be introduced into the animal using in vitro or in vivo formats. For example, the nucleic acid can be introduced into a cell in vitro and then be transferred into the animal. In another embodiment, the nucleic acid is introduced into a cell in vivo.

Animal models include, for example, animal models of RA. Several animal models of RA, and methods for generating such models, are known in the art. Such models include adjuvant-induced arthritis (AA) [see, e.g., Kong et al. (1999) Nature 4023:304-309] and collagen type II-induced arthritis [see, e.g., Tak et al. (1999) Rheumatology 38:362-369; Han et al. (1998) Autoimmunity 28:197-208; Gerlag et al. (2000) J. Immunology 165:1652-1658]. For example, experimental induction of adjuvant-induced arthritis in Lewis rats leads to severe inflammation in the bone marrow and soft tissues surrounding joints accompanied by extensive local bone and cartilage destruction, loss of bone mineral density and crippling [see, e.g., Bendele et al. (1999) Arthritis Rheum. 42:498-506].

D. Assessing the Delivery of Nucleic Acid into Cells

Microscopic and colony formation analysis methods that can be used in evaluating stable nucleic acid molecule delivery rely on manual visualization or measurement of nucleic acid molecules (e.g., a selectable marker gene) expression, which is a distinct process from delivery. Such methods are associated with time delays in obtaining an assessment of the delivery method. Microscopic techniques for visualizing chromosome or plasmid transfer using bromodeoxyuridine (BrdU) (see. e.g., Pittman et al. J Immunol Methods 103:87-92 (1987)) are time consuming, restricted by the large sample size required to detect low levels of transfer and limited by the necessity of manual scoring. Colony-forming transfection analysis can require four-to-six weeks to generate and evaluate marker-expressing transfection colonies.

In contrast, methods provided herein are based on rapid, automated, sensitive and accurate analysis procedures, such as flow cytometry, and thus do not involve any time-consuming, laborious and error-prone steps, such as manual detection of individual transfected cells by microscopic techniques. The methods make possible the analysis of nucleic acid molecule delivery data within 48 hours after transfection. Also, data collected by flow cytometry analysis is statistically superior due to the ease at which large numbers of events, e.g., nucleic acid molecule transfer, are collected. The positive values obtained in these methods are instrument derived and therefore not as susceptible to judgment errors. Thus, these methods provide for greater accuracy in assessing nucleic acid molecule delivery. In contrast, microscopic analysis is limited by the time involved for scoring positive events and sample size is restrictive.

Because the methods of monitoring nucleic acid molecule delivery detect labeled nucleic acid molecules, such as DNA, and not a reporter gene expression product, it is possible to measure absolute values of nucleic acid molecules transferred, within twenty-four hours, without being hindered by cell autofluorescence and by the problems of differentiating wild-type cells from cells expressing low levels of reporter gene products (see, e.g., Ropp et al. (1995) Cytometry 21:309-317).

1. Factors to Consider in Addressing Delivery of Nucleic Acids

Delivery of nucleic acids, including DNA, into cells is a process in which nucleic acids are transferred to the interior of a cell. Methods for the delivery of nucleic acids can be assessed in a variety of ways, including the following.

a. Transfer Efficiency

A delivery method can be assessed by determining the percentage of recipient cells in which the nucleic acids, including DNA, are present (i.e., the transfer efficiency). However, when evaluating a delivery method for the ultimate goal of generating cells that express the transferred nucleic acid, there are additional factors beyond mere presence of the nucleic acid in recipient cells that should be considered. Included among these additional factors is cell viability. When assessing a proliferating cell population, clonogenicity is the method of choice to measure viability. When the target cells population is non-dividing or slow growing, metabolic integrity can be monitored.

b. Clonogenicity

Clonogenicity represents a measure of the survivability of cells with respect to a delivery procedure, growth conditions and cell manipulations (e.g., plating). It is important to assess clonogenicity to determine whether a delivery procedure results in a sufficient number of viable cells to achieve a desired number of cells containing the transferred nucleic acid.

Clonogenicity can be expressed as a clonal fraction. The clonal fraction is an index that is calculated by multiplying two separate fractions and normalizing to a control plating efficiency correction factor (CPE). The two separate fractions that are multiplied in this calculation are the fraction of cells that survive a delivery procedure (population cell yield) and the fraction of cells that survive a plating procedure. The calculation is thus as follows:

$$Colonal Fraction = \frac{\# viable colonies after plating}{\# cells plated} \times \frac{\# cells post \text{-} tranfection}{\# cells transfected \times CPE}$$

The values used in this calculation for the number of cells post-transfection (i.e., post-delivery) and the number of colonies post-plating is based on cell or colony numbers at certain times in the process. For instance, the value for the number of cells post-transfection is representative of the number of cells at a time after nucleic acid delivery that is sufficient for the delivery process to be completed. This time can be determined empirically. Typically this time ranges from 4-48 hours and generally is about one day after transfection. Likewise, the value of the number of viable colonies post-plating is representative of the number of colonies at a time after nucleic acid delivery that is sufficient for the non-viable cells to be eliminated and the viable cells to be established as colonies. This time can be determined empirically. Typically this time ranges from that in which the average colony is made up of approximately 50 cells or generally is a time at which five cell cycles have passed.

A correction factor is included to take into account the plating efficiency of control wells, which is the ratio determined by the number of colonies counted divided by the number cells initially plated (typically 600-1000 cells). For LM(tk−) and V79-4 cells, the value of the correction factor typically ranges from about 0.7 to about 1.2 and can be, for example, 0.9.

The number of cells plated should remain constant at 1000 (simplified plating efficiency assay) done in duplicate, except in the case where the CPE is below 0.3, then number of cells seeded should be increased to a range of 5,000-50,000. If the CPE is below 0.1-0.2, then a viable fraction analysis should be considered.

c. Viable Fraction

If the target cells population is non-dividing or slowly dividing then reproductive or clonogenicity assays are not relevant. Less direct measurements of cell viability must be used to measure cell killing that monitor metabolic death rather than loss of reproductive capacity. These procedures include, for example: (1) membrane integrity as measured by dye exclusion, (2) inhibition of nucleic acid synthesis as measured by incorporation of nucleic acid precursors, (3) radioactive chromium release, and (4) MTT ASSAY (3-[4,5-dimethylthiazol-2-yl]-2,5-diphenyl-tetrazolium bromide). These methods are different from measurements of loss of proliferative capacity, as they reflect only immediate changes in metabolism, which can be reversed or delayed and hence lead to errors in estimation of cell viability. To minimize these errors, correlation of duplicate procedures is suggested.

d. Potential Transfection Efficiency (PTE) and Determination of Chromos Index (CI)

In assessing a delivery method used to transfer nucleic acids to cells with the goal of expression of the nucleic acids, including DNA, therein, it is desirable to obtain an indication of the theoretical maximum percentage of cells that are viable and contain the nucleic acid out of the total number of cells into which nucleic acids were delivered. This is referred to as the potential transfection efficiency and can be calculated from existing or historical experimental data sets and is determined as follows:

Potential Transfection Efficiency (PTE)=Transfer Efficiency×(Clonal Fraction or Viable Fraction)×correction factor (CF)

The Chromos Index (C.I.) is an effective and rapid method to determine the Potential Transfection Efficiency of a proliferating population by using experimental values of % labeled nucleic acid, such as ACes, delivery to measure transfer efficiency and clonal fraction measured using a simplified clonogenicity assay.

Chromos Index (CI)=% labeled ACes delivery×estimated Clonal fraction×CF

The values of the transfer efficiency and of the clonal fraction and viable fraction are calculated as described above. The correction factor (CF) takes into account sample size, sample time and control plating efficiency. If all these factors are constant for each variable i.e., sampling time and size then the correction factor will approach the inverse of the value for the C.P.E., i.e., such that the clonal fraction or transfer efficiency can still approach 100% even with a low CF, or in other words, if delivery and viability are 100%, then the maximum potential transfection efficiency will equal the plating efficiency of the control cells. The calculation of C.I. allows for determination of each variable optimization, with the goal being for parameters, such as transfer efficiency, clonal fraction, and CF to approach one (or 100%). If sample size or time varies for either clonal fraction or transfer efficiency, then CF represents the extrapolated value based on slope or rate of change. An application of this assessment is provided in the EXAMPLES.

A stable transfection efficiency of about 1% is in the range (1-100%) that is considered useful for the introduction of large nucleic acid molecules into target cells. It is possible, using methods provided herein, to predict which delivery methods have to be selected for achieving desired transfection efficiencies without having to grow transfectants for extended times under selective conditions and determine numbers of cells surviving selection marker expression. This analysis involves calculation of the Chromos Index (CI) which integrates a “biological” value (the clonal fraction) with a measurement of chromosomal “uptake” or transfer efficiency (percentage of cells containing delivered ACes).

2. Labeling of Nucleic Acid Molecules for Transfer

In the methods for monitoring nucleic acid molecule delivery provided herein, the nucleic acid molecules, such as DNA, to be delivered are labeled to allow for detection of the nucleic acid molecules in recipient cells after transfer into the cells. The nucleic acid molecules can be labeled by incorporation of nucleotide analogs. Any nucleic acid molecule analog that can be detected in a cell can be used in these methods. The analog is either directly detectable, such as by radioactivity, or can be detected upon binding of a detectable molecule to the analog that specifically recognizes the analog and distinguishes it from nucleotides that make up the endogenous nucleic acid molecules, such as DNA, within a recipient cell. Analogs that are directly detectable have intrinsic properties that allow them to be detected using standard analytical methods. Analogs also can be detectable upon binding to a detectable molecule, such as a labeled antibody that binds specifically to the analogs. The label on the antibody is one that can be detected using standard analytical methods. For example, the antibody can be fluorescent and be detectable by flow cytometry or microscopy.

In particular embodiments of these methods, the nucleic acid molecules, such as DNA, to be transferred is labeled with thymidine analogs, such as lododeoxyuridine (IdUrd) or Bromodeoxyuridine (BrdU). In preferred embodiments, IdUrd is used to label the nucleic acid molecules, such as DNA, to be transferred. The transferred IdUrd-labeled nucleic acid molecules, such as DNA, can be immunologically tagged using an FITC-conjugated anti-BrdU/ldUrd antibody and quantified by flow cytometry. Thus, the transfer of the labeled nucleic acid molecules, such as DNA, into recipient cells can be detected within hours after transfection.

E. Stability of Nucleic Acid Molecules to be Delivered

It is also of interest to evaluate the stability of the nucleic acid molecule, such as DNA, under the selected delivery conditions. Some delivery conditions and agents can have adverse effects on nucleic acid molecule structure. Furthermore, the labeling techniques used in certain methods of monitoring nucleic acid molecules, such as DNA, delivery can also impact nucleic acid molecules, such as DNA, structure and function.

The effects of delivery conditions on nucleic acid molecules can be assessed in a variety of ways, including microscopic analysis. In a particular exemplary analysis of the stability of artificial chromosomes, e.g., ACes, the chromosomes are exposed to the conditions of interest, e.g., IdU labeling, and analyzed under a fluorescent microscope for the ability to remain intact and condensed after incorporation of nucleotide analogs.

Methods of Monitoring Nucleic Acid Molecule Delivery and Expression

Methods of monitoring delivery of nucleic acid molecules delivery provided herein also can be combined with an assessment of nucleic acid molecule, such as DNA, expression in recipient cells to provide even further information concerning the overall process of nucleic acid molecule transfer for purposes of expression.

For example, to facilitate analysis of nucleic acid molecules, such as DNA, expression, it is desirable to include in the transferred nucleic acid molecules, such as DNA, a reporter gene that encodes a readily detected product. For direct detection, such reporter gene products include, but are not limited to green fluorescent proteins (GFP), Red Fluorescent protein (RFP), luciferases, and CAT. For indirect detection, reporter gene products include, but are not limited, to β-galactosidase and cell surface markers.

By using, for example, artificial chromosomes such as ACes containing a GFP reporter gene, such as, but are not limited to, GFP coding sequences in combination with labeling of the ACes with DNA analogs, such as IdU, delivery and expression can be rapidly and accurately monitored. For example, following the delivery of IdU-labeled GFP gene-containing ACes to target cells by any of the described methods, the cells containing the ACes are split into two populations. One population is fixed and stained for IdU and analyzed by flow cytometry to determine percentage delivery. The other population is allowed to go through 4-5 cell divisions (approximately 72 hours), and the GFP fluorescence is measured as an indication of expression.

Such studies have revealed that incorporation of the analog label does not affect GFP protein expression, which indicates that the methods can be combined to monitor delivery and early expression of the ACes, thus providing more information to rapidly evaluate the efficiency of delivery methods. The combined methods also can be used to map the biological events between the initial stages of delivery and early gene expression.

The following examples are included for illustrative purposes only and are not intended to limit the scope of the invention.

### EXAMPLE 1

### Preparation of Artificial Chromosomes

A. GFP Chromosome Contained in A9 Cell Line Plasmids

Plasmid pIRES-EGFP (see SEQ ID No. 13, plasmid obtained from Clontech, Calif., and is well known, see, e.g., U.S. Pat. Nos. 6,034,228, 6,037,133, 5,985,577, 5,976,849, 5,965,396, 5,976,796, 5,843,884, 5,962,265, 5,965,396; see, also, U.S. Pat. No. 4,937,190). This plasmid contains the internal ribosome entry site (IRES; Jackson (1990) Trends Biochem. 15:477-483; Jang et al. (1988) J. Virol. 62:2636-2643) of the encephalomyocarditis virus (ECMV) between the MCS and the enhanced green fluorescent protein (EGFP) coding region. This permits the gene of interest (cloned into the MCS) and the EGFP gene to be translated from a single bicistronic mRNA transcript. Plasmid pIRES2-EGFP is designed for selection, by flow cytometry and other methods, of transiently transfected mammalian cells that express EGFP and the protein of interest. This vector also can be used to express EGFP alone or to obtain stably transfected cell lines without drug and clonal selection.

Enhanced GFP (EGFP) is a mutant of GFP with a 35-fold increase in fluorescence. This variant has mutations of Ser to Thr at amino acid 65 and Phe to Leu at position 64 and is encoded by a gene with optimized human codons (see, e.g., U.S. Pat. No. 6,054,312). EGFP is a red-shifted variant of wild-type GFP (Yang et al. (1996) Nucl. Acids Res. 24:4592-4593; Haas et al. (1996) Curr. Biol. 6:315-324; Jackson et al. (1990) Trends Biochem. 15:477-483) that has been optimized for brighter fluorescence and higher expression in mammalian cells (excitation maximum=488 nm; emission maximum=507 nm). EGFP encodes the GFPmut1 variant (Jackson (1990) Trends Biochem. 15:477-483) which contains the double-amino-acid substitution of Phe-64 to Leu and Ser-65 to Thr. The coding sequence of the EGFP gene contains more than 190 silent base changes which correspond to human codon-usage preferences (Jang et al. (1988) J. Virol. 62:2636-2643). Sequences flanking EGFP have been converted to a Kozak consensus translation initiation site (Huang et al. (1990) Nucleic Acids Res. 18: 937-947) to further increase the translation efficiency in eukaryotic cells.

Plasmid pIRES-EGFP was derived from PIRESneo (originally called pCIN4) by replacing the neo gene downstream of the IRES sequence with the EGFP coding region. The IRES sequence permits translation of two open reading frames from one mRNA transcript. The expression cassette of pIRES-EGFP contains the human cytomegalovirus (CMV) major immediate early promoter/enhancer followed by a multiple cloning site (MCS), a synthetic intron (IVS; Huang et al. (1990) Nucleic Acids Res. 18: 937-947), the EMCV IRES followed by the EGFP coding region and the polyadenylation signal of bovine growth hormone.

Location of Features (with reference to SEQ ID No. 13): Human cytomegalovirus (CMV) immediate early promoter: 232-820;

MCS 909-974;

IVS 974-1269;

IRES of ECMV 1299-1884;

Enhanced green fluorescent protein (EGFP) gene 1905-2621;

fragment containing the bovine polyA signal 2636-2913;

Col E1 origin of replication 3343-4016; and

Ampicillin resistance gene 5026-4168

Propagation in E. coli

Suitable host strains: DH5a, HB101, and other general purpose strains. Single-stranded DNA production requires a host containing an F plasmid such as JM101 or XL1-Blue.

Selectable marker: plasmid confers resistance to kanamycin (30 μg/ml) to E. coli hosts.

E. coli replication origin: pUC

Copy number: ˜500

Plasmid incompatibility group: pMB1/ColE1

pCHEGFP2

Plasmid pCHEGFP2 was constructed by deletion of the Nsi1/SmaI fragment from pIRES-EGFP. Plasmid pIRES-EGFP contains the coding sequence for a 2.1 kB NruI/XhoI fragment of pCHEGFP2 containing the CMV promoter, synthetic intron, EGFP coding sequence and bovine growth hormone polyadenylation signal. Digestion of pIRES-EGFP with Nrul and Smal, yielded a 2.1 kb fragment. Digested DNA was fractionated by agarose gel electrophoresis, the separated band was excised and then eluted from the gel using the Qiaex 11 gel purification system (Qiagen, Mississauga, Ontario).

pFK161

Cosmid pFK161 was obtained from Dr. Gyula Hadlaczky and contains a 9 kb NotI insert derived from a murine rDNA repeat (see clone 161 described in PCT Application Publication No. WO97/40183 by Hadlaczky et al. for a description of this cosmid). This cosmid, referred to as clone 161 contains sequence corresponding to nucleotides 10,232-15,000 in SEQ ID NO. 16. It was produced by inserting fragments of the megachromosome (see, U.S. Pat. No. 6,077,697 and International PCT application No. (WO 97/40183); for example, H1 D3, which was deposited at the European Collection of Animal Cell Culture (ECACC) under Accession No. 96040929, is a mouse-hamster hybrid cell line carrying this megachromosome) into plasmid pWE15 (Stratagene, La Jolla, Calif.) as follows. Half of a 100 μl low melting point agarose block (mega-plug) containing isolated SATACs was digested with NotI overnight at 37° C. Plasmid pWE15 was similarly digested with NotI overnight. The mega-plug was then melted and mixed with the digested plasmid, ligation buffer and T4 ligase. Ligation was conducted at 16° C. overnight. Bacterial DH5α cells were transformed with the ligation product and transformed cells were plated onto LB/Amp plates. Fifteen to twenty colonies were grown on each plate for a total of 189 colonies. Plasmid DNA was isolated from colonies that survived growth on LB/Amp medium and was analyzed by Southern blot hybridization for the presence of DNA that hybridized to a pUC19 probe. This screening methodology assured that all clones, even clones lacking an insert but yet containing the pWE15 plasmid, would be detected.

Liquid cultures of all 189 transformants were used to generate cosmid minipreps for analysis of restriction sites within the insert DNA. Six of the original 189 cosmid clones contained an insert. These clones were designated as follows: 28 (˜9-kb insert), 30 (˜9-kb insert), 60 (˜4-kb insert), 113 (˜9-kb insert), 157 (˜9-kb insert) and 161 (˜9-kb insert). Restriction enzyme analysis indicated that three of the clones (113, 157 and 161) contained the same insert.

For sequence analysis the insert of cosmid clone no. 161 was subcloned as follows. To obtain the end fragments of the insert of clone no. 161, the clone was digested with NotI and BamHI and ligated with NotI/BamHI-digested pBluescript KS (Stratagene, La Jolla, Calif.). Two fragments of the insert of clone no. 161 were obtained: a 0.2-kb and a 0.7-kb insert fragment. To subclone the internal fragment of the insert of clone no. 161, the same digest was ligated with BamHI-digested pUC19. Three fragments of the insert of clone no. 161 were obtained: a 0.6-kb, a 1.8-kb and a 4.8-kb insert fragment.

The insert corresponds to an internal section of the mouse ribosomal RNA gene (rDNA) repeat unit between positions 7551-15670 as set forth in GENBANK accession no. X82564, which is provided as SEQ ID NO. 5. The sequence data obtained for the insert of clone no. 161 is set forth in SEQ ID NOS. 6-12. Specifically, the individual subclones corresponded to the following positions in GENBANK accession no. X82564 (i.e., SEQ ID NO. 5) and in SEQ ID NOs. 6-12:

| Subclone | Start | End | Site | SEQ ID No. |
|---|---|---|---|---|
| | in X82564 | | | |
| 161k1 | 7579 | 7755 | NotI, BamHI | 6 |
| 161m5 | 7756 | 8494 | BamHI | 7 |
| 161m7 | 8495 | 10231 | BamHI | 8 (shows only sequence |
| | | | | corresponding to |
| | | | | nt. 8495-8950), |
| | | | | 9 (shows only sequence |
| | | | | corresponding to |
| | | | | nt. 9851-10231) |
| 161m12 | 10232 | 15000 | BamHI | 10 (shows only sequence |
| | | | | corresponding to |
| | | | | nt. 10232-10600), |
| | | | | 11 (shows only sequence |
| | | | | corresponding to |
| | | | | nt. 14267-15000) |
| 161k2 | 15001 | 15676 | NotI, BamHI | 12 |

The sequence set forth in SEQ ID NOs. 6-12 diverges in some positions from the sequence presented in positions 7551-15670 of GENBANK accession no. X82564. Such divergence can be attributable to random mutations between repeat units of rDNA.

For use herein, the rDNA insert from the clone was prepared by digesting the cosmid with NotI and BglII and was purified as described above. Growth and maintenance of bacterial stocks and purification of plasmids were performed using standard well known methods (see, e.g., Sambrook et al. (1989) Molecular Cloning: A Laboratory Manual, 2nd Edition, Cold Spring Harbor Laboratory Press), and plasmids were purified from bacterial cultures using Midi- and Maxi-preps Kits (Qiagen, Mississauga, Ontario).

B. Preparation of the GFP, Murine A9 Cell Line

Cell Culture and Transfection

The murine A9 cell line was obtained from ATCC and cells were thawed and maintained as described below. Briefly, cells were plated at a density of 2×10⁶ cells per 15 cm tissue culture dish (Falcon, Becton Dickinson Labware, Franklin Lakes, N.J.) in growth medium containing of 90% DMEM (Canadian Life Technologies Burlington, ON) and 10% FBS (Can Sera, Rexdale ON), and were maintained at 37° C., 5% CO₂. Cultures were routinely passaged when cells reached 70%-80% confluence. Sub culturing was carried out as follows: medium was removed by aspiration, 10 ml of 1×trypsin-EDTA (Canadian Life Technologies Burlington, ON) was dispensed onto the cell monolayer and the dish gently swirled to distribute the trypsin-EDTA. Finally, the bulk of the trypsin-EDTA was removed by aspiration, and the dish placed at 37° C. for 5 minutes. To quench the trypsin-EDTA, 10 ml of growth medium was added to the dish, and the single cell suspension was transferred to a 50 ml conical tube. Cell counts were performed using a cell counting apparatus (Beckman-Coulter, Hialeah Fla.). The cells were diluted and re-plated as described above. For cryo-storage, cultures were harvested by treatment with trypsin-EDTA, counted and the cell suspension then centrifuged at 500×g for 5 minutes in a swinging bucket centrifuge. The cell pellet was resuspended in freezing medium containing 90% DMEM, 20% FBS and 10% DMSO (Sigma-Aldrich, Oakville, ON) at a density of 1×10⁷ cells/ml. One ml aliquots of the cell suspension were then dispensed into cryo-vials (Nunc, Rochester N.Y.), frozen over night in an isopropanol filled container (NUNC, Rochester N.Y.) and placed at −70° C. and then transferred to the gas phase of a liquid nitrogen freezer for long-term storage.

A9 cells were transfected using the Ca₂PO₄ co-precipitation method (see, e.g., Graham et al. (1978) Virology 52:456-457; Wigler et al. (1979) Proc. Natl. Acad. Sci. U.S.A. 76:1373-1376; and (1990) Current Protocols in Molecular Biology, Vol. 1, Wiley Inter-Science, Supplement 14, Unit 9.1.1-9.1.9). One day prior to-transfection, A9 cells were plated at a density of 2×10⁶ cells per 10 cm dish and 3 hours before transfection the medium was replaced with fresh growth medium. 140 μg of the 9 kb rDNA, NotI and 5 μg of the 2.1 kB CMV-EGFP XhoI/NruI fragments were mixed, co-precipitated and used to prepare the Ca₂PO₄ co-precipitate (Calcium Phosphate Transfection System, (Canadian Life Technologies Burlington, ON)), which was distributed onto 2 10-cm dishes of subconfluent A9 cells. The DNA-Ca₂PO₄ complexes were left on the cells for 18 hours, after which the precipitate was removed by aspiration and cells were subjected to glycerol shock for 1.5 minutes. After glycerol shock, the cell monolayers were gently washed with 2×10 ml of dPBS (Canadian Life Technologies Burlington, ON), followed by addition of 10 ml pre-warmed growth medium. Finally dishes were returned to the incubator and were maintained at 37° C., 5% CO₂. After 3 hours recovery, each dish was passaged onto 3×15 cm tissue dishes

GFP fluorescence of cultures was monitored visually during culture using an inverted microscope equipped with epifluorescence illumination (Axiovert 25; Zeiss, (North York ON)) and #41017 Endow GFP filter set (Chroma Technologies, Brattleboro, Vt.). Enrichment of GFP expressing populations was carried out as described below.

Enrichment of GFP Expressing Cell Populations by Fluorescence Activated Cell Sorting

Cell sorting was carried out using a FACS Vantage flow cytometer (Becton Dickinson Immunocytometry Systems, San Jose, Calif.) equipped with turbo-sort option and 2 Innova 306 lasers (Coherent, Palo Alto Calif.). For cell sorting a 70 μm nozzle was used. The sheath buffer was changed to PBS (maintained at 20 p.s.i.). GFP was excited with a 488 nm Laser beam and excitation detected in FL1 using a 500 EFLP filter. Forward and side scattering was adjusted to select for viable cells. Only viable cells were then analyzed for GFP fluorescence. Gating parameters were adjusted using wild type A9 cells as negative control and GFP CHO cells as positive control.

For the first round of sorting, A9 cells were harvested 4 days post-transfection, resuspended in 10 ml of growth medium and sorted for GFP expressing populations using parameters described above. GFP positive cells were dispensed into, a volume of 5-10 ml of growth medium supplemented with 1×penicillin/streptomycin (Canadian Life Technologies Burlington, ON) while non-expressing cells were directed to waste. The expressing cells were further diluted to 50 ml using the same medium, plated onto 2×15 cm dishes and cultured as described in the previous section. When the sorted populations reached confluence they were re-sorted to enrich for GFP expressing cells. A total of 4 sequential sorts were carried out, achieving enrichments of as high as 89% GFP expressing cells after the final sort. The final GFP expressing populations were expanded for cryo-preservation and for fluorescence in-situ hybridization screening (see below). Single cell clones were established from populations of interest by using the flow cytometer to direct GFP expressing single cells to individual wells of 96 well plates. These were cultured as described above.

Fluorescence In-Situ Hybridization

Fluorescence In-Situ Hybridization (FISH) screening was carried out on GFP enriched populations and single cell clones to detect amplification and/or artificial chromosome formation. Preparation of metaphase spreads and hybridizations were performed (see, Telenius et al. (1999) Chromosome Res 7:3-7). Probes used include pSAT 1, which recognizes the mouse major repeat (see, e.g., Wong et al. (1988) Nucl. Acids Res. 16:11645-11661), pFK161, which hybridizes to the mouse rDNA-containing regions and a PCR generated probe against the mouse minor repeat.

C. Purification of Artificial Chromosomes by Flow Cytometry and Preparation of DNA from Flow Sorted Chromosomes

Artificial chromosomes were purified from the host cell by flow cytometry (see de Jong (1999) Cytometry 35:129-133). Briefly, purification was performed on FACS Vantage flow cytometer (Becton Dickinson Immunocytometry Systems, San Jose, Calif.) equipped with a Trubo-Sort Option and two Innova 306 lasers (Coherent, Palo Alto, Calif.). The Turbo Sort Option modification include increasing the maximum system pressure from 20 lb/in² to 60 lb/in², increasing the drop drive frequency from 50,000 drops/s to a maximum of 99,000 drops/s and increasing the deflection plate voltages from a maximum 6,000 V to 8,000 V. Other modifications are made to the instrument to accommodate the higher pressures. Hoechst 35258 was excited with the primary UV laser beam, and excitation detected in FLI by using 420 nm hand-pass filter. Chromomycin A3 was excited by the second laser set at 458 nm and fluorescence detected in FL 4 by using a 475 nm long-pass filter. Both lasers had an output of 200 mW. Bivariate distributions (1,024×10²⁴ channels) were accumulated during each sort. For all chromosome sorts, the sheath pressure was set at 30 lb/in² and a 50 μm diameter nozzle was installed. A drop delay profile was performed every morning and repeated after any major plug. Alignment of the instrument was performed daily by using 3.0 pm diameter Sphero rainbow beads (Spherotech, Libertyville, Ill.). Alignment was considered optimized when a CV of 2.0% or less was achieved for FL1 and FL4.

Condensing agents (hexylene glycol, spermine and spermidine) were added to the sheath buffer to maintain condensed chromosomes after sorting. The sheath buffer contains 15 nM Tris HCl, 0.1 mM EDTA, 20 mM NaCl, 1% hexylene glycol, 100 mM glycine, 20 μM spermine and 50 μM spermidine. The sorted chromosomes were collected in 1.5 ml screw-capped Eppendorf tubes at 4° C. at a concentration of approximately 1×10⁶ chromosomes/ml, which were then stored at 4° C.

For preparation of purified genomic DNA, sorted chromosome samples were brought to 0.5% SDS, 50 mM EDTA and 100 μpg/ml Proteinase K, then incubated for 18 hours at 50° C. 1 μl of a 20 mg/ml glycogen solution (Boehringer Mannheim) was added to each sample, followed by extraction with an equal volume of Phenol: Chloroform: isoamyl Alcohol (25:24:1). After centrifugation at 21,000×g for 10 min, the aqueous phases were transferred to fresh microfuge tubes and were re-extracted as above. 0.2 volumes of 10 M NH₄OAC, 1 μl of 20 mg/ml glycogen and 1 volume of iso-propanol were added to the twice extracted aqueous phases which were then vortexed and centrifuged for 15 minutes at 30,000×g (at room temperature). Pellets were washed with 200 μl of 70% ethanol and re-centrifuged as above. The washed pellets were air-dried then resuspended in 5 mM Tris-Cl, pH 8.0 at 0.5-2×10⁶ chromosome equivalents/μl.

PCR was carried out on DNA prepared from sorted chromosome samples essentially as described (see, Co et al. (2000) Chromosome Research 8:183-191) using primers sets specific for EGFP and RAPSYN. Briefly, 50 μl PCR reactions were carried out on genomic DNA equivalent to 10,000 or 1000 chromosomes in a solution containing 10 mM Tris-Cl, pH 8.3, 50 mM KCl, 200 μM dNTPs, 500 nM of forward and reverse primers, 1.5 mM MgCl₂, 1.25 units Taq polymerase (Ampli-Taq, Perkin-Elmer Cetus, Calif.). Separate reactions were carried out for each primer set. The reaction conditions were as follows: one cycle of 10 min at 95° C., then 35 cycles of 1 min at 94° C., 1 min at 55° C., 1 min at 72° C., and finally one cycle of 10 min at 72° C. After completion the samples were held at 4° C. until analyzed by agarose gel electrophoresis using the following primers (SEQ ID Nos. 1-4, respectively):

EGFP forward primer
5′-cgtccaggagcgcaccatcttctt-3′;
EGFP reverse primer
3′-atcgcgcttctcgttggggtcttt-3′;
RAPSYN forward primer
5′-aggactgggtggcttccaactcccagacac-3′; and
RAPSYN reverse primer
5′-agcttctcattgctgcgcgccaggttcagg-3′.

All primers were obtained from Canadian Life Technologies, Burlington, ON.

### EXAMPLE 2

Preparation of Cationic Vesicles

Vesicles were prepared at a lipid concentration of 700 nmol/ml lipid (cationic lipid/DOPE 1:1) as follows. In a glass tube (10 ml) 350 nmol cationic lipid (SAINT-2) was mixed with 350 nmol dioleyl-phosphatidylethanolamine (DOPE), both solubilized in an organic solvent (Chloroform, Methanol or Chloroform/Methanol 1:1, v/v). Dioleylphosphatidylethanolamine(DOPE; Avanti Polar Lipids, Alabaster, Ala.) forms inverse hexagonal phases in a membrane and weakens the membrane. Other effectors that can be used are cis-unsaturated phosphatidylethanolamines, cis-unsaturated fatty acids and cholesterol. Cis-unsaturated phosphatidylcholines are less effective.

The solvent was evaporated under a stream of nitrogen (15 min/250 μl solvent at room temperature). The remaining solvent was removed totally by drying the lipid for 15 min in a desiccator under high vacuum from a vacuum pump. To the dried mixture was added 1 ml ultrapure water. This was vortexed vigorously for about 5 min. The resulting solution was sonicated in an ultrasonication bath (Laboratory Supplies Inc. NY) until a clear solution was obtained. The resulting suspension contained a population of unilamellar vesicles with a size distribution between 50 to 100 nm.

### EXAMPLE 3

Preparation of Cationic Vesicles via Alcoholic Injection

In a glass tube (10 ml) 350 nmol cationic lipid (Saint-2) was mixed with 350 nmol DOPE, both solubilized in an organic solvent (chloroform, methanol or chloroform/methanol 1/1). The solvent was evaporated under a stream of nitrogen (15 min/250 μl solvent at room temperature). The remaining solvent was removed totally by drying the lipid for 15 min under high vacuum. This was then reconstituted in 100 μl pure ethanol.

### EXAMPLE 4

Transfection of beta ACes into V79-4 Cell Line

Transfection Procedure for Various Transfection Agents

All compounds were tested in a Chinese Hamster lung fibroblast line (V79-4, ATCC number CCL-39). Approximately 17 hours (2 cell doublings) prior to transfection, exponentially growing cells were trypsinized and plated at 250,000 cells per well into a 6 well petri dish with Dulbecco's Modified Eagle Medium (Life Technologies, Burlington, ON) and supplemented with 10% FBS (Can Sera, Rexdale ON)). At the time of transfection, the number of cells per well was estimated to be approximately 1 million. For transfection, each individual manufacturer's protocol for complexing to naked DNA was followed, with the exception that the amount of transfection agent used was varied, to reflect the different amount and type of DNA present, as well as the different ionic strength of the complexing. One million ACes (in a volume of 800 μl) were typically combined with the transfection agent in a wide range of concentrations (between 5 times and 100 times the lowest manufacturers suggested concentration). The ACes/transfection mixture was allowed to complex for the time recommended by the manufacturer, in volumes ranging from 0.8 ml to 1.9 ml; some manufacturers recommend adding media to the complexing reaction. The complexed mixture was then applied to the recipient cells and transfection allowed to proceed according to the manufacturer's protocol. Details on the various conditions used with different agents are presented in Table 1.

Transfection Procedure for Superfect Agent

Superfect was tested in a Chinese Hamster lung fibroblast line (V79-4, ATCC number CCL-39). Approximately 17 hours (2 cell doublings) prior to transfection, exponentially growing cells were trypsinized and plated at 250,000 cells per well into a 6 well petri dish with Dulbecco's Modified Eagle Medium (Life Technologies, Burlington, ON) and supplemented with 10% FBS (Can Sera, Rexdale ON). One million ACes in 800 μl of sort buffer was complexed to 101 μl of Superfect reagent. Complex was incubated at room temperature for 10 minutes. At the time of transfection, the number of cells per well was estimated to be approximately 1 million. Media was removed from wells and 600 μl of DMEM and 10% FBS was added. Superfect:ACes complex was added to the wells drop-wise and allowed to incubate for 3 hours at 37° C. After incubation, transfected cells were trypsinized and transferred to 15 cm dishes with 25 ml DMEM and 10% FBS and allowed to attach for 24 hours. After 24 hours, selection medium containing of 0.7 mg/ml hygromycin B was added to each well. The selection medium was changed every 2-3 days. After 10-12 days colonies were screened for Beta-galactosidase expression and/or FISHed for detection of intact chromosome.

Example of Application of the Determination of the Chromos Index

Approximately 1×10⁶ V79-4 cells were transfected with 1×10⁶ IdUrd-labeled ACes complexed with a delivery agent (i.e., Lipofectamine PLUS and Lipofectamine or Superfect). The transfected cells were then fixed in ethanol. Fixed cells were denatured and exposed to FITC-conjugated antibody that specifically binds to BrdU/IdUrd-labeled nucleic acids.

The percentage of transfected cells containing IdUrd-labeled ACes was determined using flow cytometry and collecting FITC fluorescence. Data were accumulated to form bivariate channel distribution showing forward scatter versus green fluorescence (IdUrd-FITC). The fluorescence level at which cells were determined to be positive was established by visual inspection of the histogram of negative control cells such that the gate for the negative cells was set such that 1% appeared in the positive region.

The number of cells recovered at 24 hours post-transfection was determined by counting an aliquot using a Coulter Counter. To determine the control plating efficiency of a recipient cell line, the untreated cells were plated at 600-1000 cells per 10 cm petri dish in growth medium and left stationary in a 5% CO₂ incubator at 37° C. for approximately five cell cycles or until average colony was made up of 50 cells. At this point the number of viable colonies was determined. The treated cells were seeded at 1000 cells if the CPE is above 0.1-0.2. If the CPE is low then the seeding density is increased to 5,000-50,000 cells per dish.

### EXAMPLE 5

Ultrasound Mediated Transfection of LMTK(−) Cells with Lipofectamine

LM(tk−) cells were grown at 37° C., 5% CO₂, in DMEM with 4500 mg/L D-glucose, L-glutamine, pyridoxine hydrochloride and 10% Fetal Bovine Serum. The corner wells of a 12-well dish were seeded with 200,000 cells per well (this is to ensure no interference from the ultrasound waves from other wells) 24 hours before use.

The GFP chromosomes were counted to verify approximately 1×10⁶ ACes per ml. The chromosomes were resuspended in the tube by flicking. Ten μl of chromosome suspension was removed and mixed with an equal volume of 30 mg/ml PI (propidium iodide) stain. Eight μl of the stained chromosomes was loaded onto a Petroff Hausser counting chamber and the chromosomes were counted.

The medium was removed from the cells, and the cells were washed twice with HBSS (without phenol red, Gibco BRL) warmed to 37° C. 500 μl of the warmed HBSS was added to each well of cells (1 μl) LipofectAMINE (Gibco BRL) was added to each well. The plates were then sealed with parafilm tape and shaken gently at 20 rpm at room temperature for 30 minutes (Stagger plates—10 minutes for ease of handling).

After incubation, Ultrasound gel (Other-Sonic Generic Ultra sound transmission gel, Pharmaceutical Innovations, Inc., Newark, N.J.) was applied to the 2.5 cm sonoporator head. Ultrasound was applied with an ImaRX Sonoporator 100 at an output energy of 2.0 Watt/cm², for 60 seconds, through the bottom of the plate of cells. After ultrasound of the well one chromosome per seeded cell (2×10⁵) or 200 μl GFP ACes in sheath buffer (15 nM Tris HCl, 0.1 mM EDTA, 20 mM NaCl, 1% hexylene glycol, 100 mM glycine, 20 μM spermine and 50 μM spermidine) are added immediately to the well. (Repeat until all samples on the plate requiring ultrasound have been treated). The plate was then sealed once more with parafilm tape and shaken gently (20 rpm) for 1 hour at room temperature.

After the incubation, 1 ml (DMEM with 4500 mg/L D-glucose, L-glutamine and pyridoxine hydrochloride, 10% Fetal Bovine Serum, and a lx solution of penicillin and streptomycin from a 10000 units/ml penicillin and 10000 mg/ml Streptomycin, 100×stock solution) was added to each well and the cells were incubated 18-24 hours at 37° C.

The cells in the plates were then washed with antibiotic containing medium and 2 ml of medium was placed in each well. The cells continued to be incubated at 37° C. with 5% CO₂ until 48 hours after transfection/sonoporation. The cells were then trypsinized and resuspended at a concentration of 1×10⁶ in DMEM to be analyzed by flow cytometry.

Results: Flow analysis was performed on a FACS Vantage (BDIS, San Jose, Calif.) equipped with a turbo-sort option and two inova 305 lasers (Coherent, Palo Alto, Calif.). The GFP signal excitation is at 488 nm and the emission detected in FL1 using a 500 nm long pass filter. Analysis of the transfected cells generated populations of GFP positive cells ranging from 13-27%. Non-sonoporated control value was 5%.

### EXAMPLE 6

Ultrasound Mediated Transfection with Saint-2

A. Ultrasound Mediated Transfection of CHO-KI Cells with Saint-2

CHO-KI cells were grown at 37°, 5% CO₂, in CHO-S-SFM 2 Medium, (Gibco BRL, Paisley, UK). Between 2×10⁵ and 5×10⁵ cells were plated onto sterile glass slides in a 12 well plate 24 h before usage.

Transfection of the cells was performed as follows. The medium was removed from the cells, and the cells were washed twice with HBSS (Hanks balanced salt solution without Phenol Red (Gibco BRL, UK)) at 37° C. Then 500 μl HBSS at 37° C. was added per well, followed by 10 μl of the freshly prepared vesicle solution (prepared in Example 2) to yield a final concentration of 23.3 nmol/ml.

Alternatively, the medium was removed from the cells, and the cells were washed twice with HBSS. 500 μl HBSS/lipid solution at 37° C. was added to each well. The HBSS/lipid solution was prepared by adding 1 μl ethanolic lipid solution (prepared as described above) to 500 μl HBSS under vigorous vortexing. The plates were then sealed with parafilm tape and shaken gently at room temperature for 30 min. After incubation, ultrasound was applied at an output energy of 0.5 Watt/cm² for 60 sec through the bottom of the plate to the cells. The ultrasound was mediated by an ultrasound gel (Aquasonic 100, Parker, N.J.) between transducer and plate. The ultrasound was applied with an ImaRx Sonoporator 100. Immediately after applying ultrasound one GFP chromosome per seeded cell (2×10⁵-5×10⁵) (prepared in Example 1) was added. The plate was then sealed again and shaken gently for 1 h at room temperature. After the incubation 1 ml medium (CHO-S-SFM 2 with 10% Fetal Calf Serum, 10000 μg/ml Penicillin and 10000 μg/ml Streptomycin Gibco BRL, Paisley, UK) was added to each well and the cells were incubated for 24 h at 37° C. The cells were then washed with medium, 1 ml medium was added and the cells were incubated at 37° for another 24 h. Detection of expressed genes was then assayed by microscopy or detection of the transferred chromosome by FISH analysis. The negative control was performed in the same way, but with no chromosomes added to the cells.

Results

After transfection, using visual inspection, 30% of the cells remained on the glass slide of which 10% were positive for green fluorescent protein expression after 48 hours (3% of original population). After culturing for two weeks, FISH was performed on the cells and 1.4% of the cells contained an intact artificial chromosome.

B. Ultrasound Mediated Transfection of Hep-G2 Cells with Saint-2

Hep-G2 cells were grown at 37° C., 5% CO₂, in DMEM with 4500 mg/l Glucose, with Pyridoxine/HCl, 10% Fetal Calf Serum, 10000 μg/ml Streptomycin and 1000 μg/ml Penicillin. Between 2×10⁵ and 5×10⁵ cells were plated onto sterile glass slides in a 12 wells plate 24 hours before usage.

Cells were transfected with GFP chromosomes using the procedure of Example 6A except that the CHO-KI medium was replaced with Hep-G2 medium.

Results

After transfection, 30% of the cells remained on the glass slide. 80% of these cells were positive for green fluorescent protein expression.

C. Ultrasound Mediated Transfection of A9 Cells with Saint-2

A9 cells were grown at 37° C., 5% CO₂, in DMEM with 4500 mg/l Glucose, with Pyridoxine/HCL, 10% Fetal Calf Serum, 10000 μg/ml Streptomycin and 10000 μg/ml Penicillin (GIBCO BRL, Paisley, UK). Between 2×10⁵ and 5×10⁵ cells were plated onto sterile glass slides in a 12 well plate 24 h before usage.

Cells were transfected with GFP chromosomes using the procedure of Example 6A except that CHO-KI medium was replaced with A9 medium.

Results

After transfection, 30% of the cells remained on the glass of which 50% were positive for green fluorescent protein expression.

### EXAMPLE 7

Delivery of ACes into Synoviocytes, Skeletal Muscle Fibroblasts and Skin Fibroblasts

A mammalian (murine) ACes artificial chromosome (˜60 Mb) containing primarily murine pericentric heterochromatin, and including a reporter gene (lacZ) and a hygromycin B selectable marker gene, prepared as described in U.S. Pat. Nos. 6,025,155 and 6,077,697 and PCT Application Publication No. WO 97/40183 was delivered into primary rat fibroblast-like synoviocytes, rat skin fibroblasts and a rat skeletal muscle fibroblast cell line (L8 cells; ATCC Accession No. CRL-1769). Prior to delivery, the ACes were labelled with iododeoxyuridine (IdUrd) as described herein.

Preparation of cells

Primary fibroblast-like synoviocytes and rat skin fibroblasts were obtained from rats using standard methods [see, e.g., Aupperle et al (1999) J. Immunol. 163:427-433 and Alvaro-Garcia et al. (1990) J. Clin. Invest. 86:1790]. Such methods include isolation of synoviocytes from rodent knees generally by removal of skin and muscle, followed by mincing of knee joint tissue. The minced tissue is then incubated with collagenase, filtered through nylon mesh and washed extensively. Cells can be cultured overnight, after which time non-adherent cells are removed. Adherent cells can be cultured and passaged by replating at a dilution when the cultures reach confluence. The cells were plated at 50,000-75,000 cells per 6-well dish in media containing low glucose DMEM, 1-glutamine, penicillin/streptomycin and 20% FBS. The cells were grown in a 5% CO₂ incubator at 37° C. for 3-5 days until approximately 80% confluence or 500,000 cells per well.

Transfection of Cells with ACes

One million IdUrd-labelled ACes were complexed with 2, 5 or 10 μl of Superfect (Qiagen) or Lipofectamine Plus (Life Technologies; Gibco) as follows. Complexing with Superfect was conducted for 10 minutes at room temperature. For complexing with Lipofectamine Plus, the indicated amounts of PLUS reagent were added to 1 million ACes and complexed at room temperature for 15 minutes. Next the indicated amounts of Lipofectamine were added into 200 μl of low glucose DMEM (no FBS) and combined with the ACes/PLUS complex for 15 minutes at room temperature. The complexed ACes were then added dropwise to the cells in 600 μl media (final volume of approximately 1.4 ml). After 3 hrs at 37° C. in a 5% CO₂ incubator, a total volume of 3 ml of culture media (low glucose DMEM, I-glutamine, penicillin/streptomycin and 20% FBS) was added. After 24-48 hrs, the cells were trypsinized to form a single cell suspension, centrifuged to remove the supernatant and then fixed in cold 70% ethanol for a minimum of one hour. An aliquot of the fixed cells was saved for microscopic analysis.

FITC-Conjugated Antibody Labelling of ACes

Following transfection, the ACes were labelled with FITC-conjugated antibody that specifically binds to BrdU- or IdUrd-labelled nucleic acids and the cells were analyzed by FACs for FITC fluorescence and microscopic staining. Fixed cells were denatured in 2N HCl and 0.5% Triton-X for 30 minutes at room temperature. After denaturation, the cells were neutralized by a series of wash steps at 4° C. To minimize background staining, the sample was resuspended in PBS and 4% FBS or BSA and 0.1% Triton-X (blocking buffer) for a minimum of 15 minutes. The cells were then pelleted and exposed for 2 hours at room temperature to FITC-conjugated antibody. After the cells were washed with blocking buffer, the sample was ready for flow cytometry analysis. Samples for microscopic analysis were dried on slides and the above staining protocol was followed, except that BrdU/IdUrd antibody was diluted 1/5 and exposed to cells for 24 hours.

Results

The delivery of intact ACes was detected within 24 to 48 hours post transfection. The number of cells recovered at 24 hours post-transfection was determined by counting an aliquot using a Coulter Counter. To determine the control plating efficiency of a recipient cell line or plating efficiency of the transfected cells, the cells were plated at 1000-10,000 cells per 10 cm petri dish in growth medium and left stationary in a 5% CO₂ incubator at 37° C. for 10 days. At that point, the number of viable colonies was determined. The normalized plating efficiency was calculated as described herein.

When Superfect was used as a delivery agent, the percent delivery into fibroblast-like synoviocytes as determined by flow cytometry ranged from ˜24% to ˜66.3%. The normalized % plating efficiency was ˜36% when 2 μl of Superfect was used and ˜16% when 5 μl of Superfect was used. Higher doses of Superfect were associated with toxicity and multiple ACes per cell as compared to lower doses. When Lipofectamine Plus was used as a delivery agent, the percent delivery into fibroblast-like synoviocytes as determined by flow cytometry ranged from ˜11% to ˜27% with percent delivery increasing with increasing doses of agent.

L8 and rat skin fibroblasts (RSF) that had been transfected with ACes were grown under hygromycin B selection and analyzed for lacZ expression. While in this example, a hygromycin selection gene was included in the ACes, there are numerous other selectable marker genes that can be used in connection with the transfer of heterologous nucleic acids into cells when it is desirable to include such genes. Such selection systems are known to those of skill in the art. A choice of selectable marker gene can, for instance, take into account the level of toxicity of the selection agent on the host cell for transfection. Identification of an appropriate selectable marker gene is routine employing the guidance provided herein.

Clones of L8 cells and RSF cells expressing lacZ were identified. These results demonstrate that IdUrd-labelled ACes can be delivered efficiently into primary cells as well as cell lines and that transgenes contained in the ACes are expressed in the transfected cells.

### EXAMPLE 8

Ex vivo transfer of reporter genes into rat joints

To examine transfer of a heterologous gene into an in vivo environment and expression of the gene in vivo, L8 cells transfected with ACes as described above were injected into the ankle joint of rats with adjuvant-induced arthritis. On day 0, adjuvant induction of arthritis was performed on Lewis rats. Methods for adjuvant induction of arthritis in animal models are known in the art [see, e.g., Kong et al. (1999) Nature 4023:304-309]. In one exemplary protocol for adjuvant induction of arthritis, Lewis rats are immunized at the base of the tail with 1 mg Mycobacterium tuberculosis H37 RA (Difco, Detroit, Mich.) in 0.1 ml mineral oil on day 0. Paw swelling typically begins around day 10.

On day 12, intra-articular injection of transfected L8 cells (˜0.7×10⁶ cells) or untransfected control cells into the right ankle joint was performed. On day 14, the rats were sacrificed in order to analyze the joints for the presence of transplanted transfected L8 cells.

Different tissues of the sacrificed rats were examined by RT-PCR analysis for the presence of lacZ mRNA. Total mRNA was extracted from the tissues and RT-PCR was performed using primers specific for the lacZ gene. The an amplification product was detected only in the synovium, and not in the other tissues (liver, kidney, heart, spleen and lung). Synovium from the sacrificed rats was also analyzed by in situ enzymatic staining X-gal staining for β-gal activity. After snap freezing of the synovium, 8 μm and 20 μm sections were cut, counter-stained with Maner's hematoxylin, and analyzed for blue staining of the cells. Staining was detected in synovium injected with L8 cells that had been transfected with ACes but not in synovium injected with untransfected cells. These results demonstrate successful ex vivo gene transfer in a rat adjuvant arthritis model using ACes containing a marker gene and thus the feasibility of treating arthritis and other connective tissue diseases using ACes as non-viral vectors for gene therapy.

### EXAMPLE 9

A Flow Cytometry Technique for Measuring Delivery of Artificial Chromosomes

Production cells lines (see Example 1) were grown in MEM medium (Gibco BRL) with 10% fetal calf serum (Can Sera, Rexdale ON) with 0.168 μg/ml hygromycin B (Calbiochem, San Diego, Calif.). lododeoxy-uridine or Bromodeoxyuridine was added directly to culture medium of the production cell line (CHO E42019) in the exponential phase of growth. Stock lododeoxyuridine made in tris base pH 10. Bromodeoxyuridine stocks in PBS. Final concentrations of 0.05-1 μM for continuous label of 20-24 hours of 5-50 μM with 15 minute pulse. After 24 hours, exponentially growing cells were blocked in mitosis with colchicine (1.0 μg/ml for 7 hours before harvest. Chromosomes were then isolated and stained with Hoechst 33258 (2.5 μg/ml) and chromomycin A3 (50 μg/ml). Purification of artificial chromosomes was performed using a FACS Vantage flow cytometer (Becton Dickinson Immunocytometry systems, San Jose, Calif.). Chromomycin A3 was excited with the primary laser set at 457 nm, with emission detected using 475 nm long pass filter. Hoechst was excited by the secondary UV laser and emission detected using a 420/44 nm band-pass filter. Both lasers had an output of 150 mW. Bivariate distribution showing cell karyotype was accumulated from each sort. ACes were gated from other chromosomes and sorted. Condensing agents (hexylene glycol, spermine, and spermidine) were added to the sheath buffer to maintain condensed intact chromosome after sorting. IdU labeling index of sorted chromosomes was determined microscopically. An aliquot (2-10 μl) of sorted chromosomes was fixed in 0.2% formaldehyde solution for 5 minutes before being dried on clean microscopic slide. The microscope sample was fixed with 70% ethanol. The air-dried slide was denatured in a coplin jar with 2N HCI for 30 minutes at room temperature and was washed 2-3 times with PBS. Non-specific binding was blocked with PBS and 4% BSA or serum for minimum of 10 minutes. A 1/5 dilution of FITC conjugated IdU/BrdU antibody (Becton Dickinson) with a final volume of 60-100 μl was applied to the slide. Plastic strips, Durra seal (Diversified Biotech, Boston, Mass.), were overlaid on slides, and slides were kept in the dark at 4%C in a humidified covered box for 8-24 hours. DAPI (Sigma) 1 μg/ml in Vectorshield was used as counterstain. Fluorescence was detected using Zeiss axioplan 2 microscope equipped for epiflorescence. A minimum of 100 chromosomes was scored for determining % labeled. Unlabeled chromosomes were used as negative control.

The day before the transfection, trypsinize V79-4 (Chinese Hamster Lung fibroblast) cells and plate at 250,000 into a 6 well petri dish in 4 ml DMEM (Dulbecco's Modified Eagle Medium, Life Technologies) and 10% FBS (Can Sera Rexdale ON). The protocol was modified for use with LM (tk−) cell line by plating 500,000 cells. Lipid or dendrimer reagent was added to 1×10⁶ ACes sorted in ˜800 μl sort buffer. Exemplary protocol variations are set forth in Table 1. Chromosome and transfection agents were mixed gently. Complexes were added to cells drop-wise and plate swirled to mix. Plates were kept at 37° C. in a 5% CO₂ incubator for specified transfection time. The volume in a well was then made up to 4-5 ml with DMEM and 10% FBS. Recipient cells were left for 24 hours at 37° C. in a 5% CO₂ incubator. Trypsinize transfected cells. Samples to be analyzed for IdU labeled chromosome delivery are fixed in cold 70% ethanol and stored at −20° C., to be ready for IdU antibody staining. Samples to be grown for colony selection are counted and then transferred to 10-cm dishes at densities of 10,000 and 100,000 cells in duplicate with remaining cells put in a 15 cm dish. After 24 hours, selection medium containing of DMEM and 10% FBS with 0.7 mg/ml hygromycin B, # 400051 (Calbiochem San Diego, Calif.) is added. Selection medium is changed every 2-3 days. This concentration of hygromycin B kills the wild type cells after selection for 7 days. At 10-14 days, colonies were expanded and then screened by FISH for intact chromosome transfer and assayed for beta galactosidase expression.

TABLE 1
Delivery Transfection Protocols

| | | | | | Medium (ml) | |
|---|---|---|---|---|---|---|
| | | | Complexing | | added to | Transfection |
| | | Pre treatment | time | Added to | wells before | time |
| Agent | Dilution Stock | of ACes | (minutes) | complexes | complexes | (hours) |
| CLONFECTIN | 2-8 μg in | | 20 | 1.8 ml of | | 4 |
| | NaCl-HEPES | | | serum free | | |
| CYTOFECTENE | | | 10-20 | 200 μl of 50% | | 24 |
| | | | | FBS plus | | |
| | | | | DMEM | | |
| ENHANCER + | | Enhancer 5 | 10 | | 1.2 | 3 |
| EFFECTENE | | minutes | | | | |
| (1:5 ratio) | | | | | | |
| EU-FECTIN-1 to | | | 5-10 | | | 6 |
| 11 | | | | | | |
| FUGENE 6 | 0.5-6 μl to | | 15-45 | | | 4 |
| | final volume | | | | | |
| | of 100 μl in | | | | | |
| | serum free | | | | | |
| | medium | | | | | |
| GENEPORTER 2 | 2.5 μl added | | 2-10 | | | 2-4 |
| | to 150 μl of | | | | | |
| | serum free | | | | | |
| | medium | | | | | |
| LIPOFECTAMINE | | | 15 | | | 3 |
| LIPOFECTAMINE | | | 20 | | 2.5 | 5 |
| 2000 | | | | | | |
| METAFECTENE | diluted into | | 15-45 | | 0.8 | 6 |
| | 60 μl serum | | | | | |
| | free medium | | | | | |
| PLUS + | | PLUS and 200 | 15 | | | 3 |
| LIPOFECTAMINE | | μl of DMEM | | | | |
| (1:1 and 3:2 | | for 15 minutes | | | | |
| ratio) | | | | | | |
| SUPERFECT | | | 10 | | 0.6 | 3 |

IdU Antibody Labeling

A standard BrdU staining flow cytometry protocol (Gratzer et al. Cytometry (1981);6:385-393) was used except with some modifications at the neutralization step, the presence of detergent during denaturation and the composition of blocking buffer. Between each step samples are centrifuged at 300 g for 7-10 minutes and supernatant removed. Samples of 1-2 million cells are fixed in 70% cold ethanol. Cells are then denatured in 1-2 ml of 2N HCL plus 0.5% triton X for 30 minutes at room temperature. Sample undergoes 3-4 washes with cold DMEM until indictor is neutral. Final wash with cold DMEM plus 5% FBS. Blocking/permeabilization buffer containing PBS, 0.1% triton X and 4% FBS is added for 10-15 minutes before pelleting sample by centrifugation. Add 20 μl of IdU/BrdU FITC conjugated B44 clone antibody (Becton Dickinson Immunocytometry Systems, San Jose, Calif.) to pellet and leave for 2 hours at room temperature in the dark with agitation every 30 minutes. Wash cells with block/permeabilization buffer and resuspend in PBS for flow analysis.

Flow Cytometry Detection of Fluorescent IdUrd-Labeled ACes

Percentage of transfected cells containing IdU labeled ACes was determined using a flow cytometry with an argon laser turned to 488 nm at 400 mW. FITC fluorescence was collected through a standard FITC 530/30-nm band pass filter. Cell populations were gated on the basis of side scatter versus forward scatter to exclude debris and doublets. Data was accumulated (15,000 events) to form bivariate channel distribution showing forward scatter versus green fluorescence (IdU-FITC). The fluorescence level at which cells were determined to be positive was established by visual inspection of the histogram of negative control cells, such that approximately 1% appeared in the positive region.

Results:

The transfection delivery results of IdU labeled ACes are set forth in Table 2.

TABLE 2

| | DOSE | DELIVERY |
|---|---|---|
| | Microliters agent | % IdU positive |
| COMPOUND | added per 1 | (24 hours) |
| CLONFECTIN | 6 | 0.61 |
| CYTOFECTENE | 8 | 14.67 |
| ENHANCER + EFFECTENE | 1.6, 10 | 17.08 |
| (1:5) | | |
| EU-FECTIN-1 | 10 | 4.57 |
| EU-FECTIN-2 | 5 | 0.14 |
| EU-FECTIN-3 | 10 | 0.69 |
| EU-FECTIN-4 | 10 | 0.24 |
| EU-FECTIN-5 | 10 | 0.41 |
| EU-FECTIN-6 | 10 | 0.46 |
| EU-FECTIN-7 | 10 | 1.21 |
| EU-FECTIN-8 | 10 | 1.58 |
| EU-FECTIN-9 | 10 | 0.6 |
| EU-FECTIN-10 | 10 | 0.77 |
| EU-FECTIN-11 | 5 | 1 |
| FUGENE | 8 | 0.49 |
| GENEPORTER | 5 | 22.12 |
| LIPOFECTAMINE | 25 | 17.81 |
| LIPOFECTAMINE 2000 | 30 | 10.96 |
| PLUS + LIPOFECTAMINE | 12, 12 | 12.2 |
| (1:1) | | |
| PLUS + LIPOFECTAMINE | 24, 16 | 26.97 |
| (3:2) | | |
| METAFECTENE | 10 | 14.14 |
| SUPERFECT | 2 | 27.67 |

Since modifications will be apparent to those of skill in this art, it is intended that this invention be limited only by the scope of the appended claims.

1. A synoviocyte comprising heterologous nucleic acid that comprises at least about 0.5 megabase pairs (Mbase).

2. The synoviocyte of claim 1, wherein the heterologous nucleic acid is greater than at least about 0.6 megabase in size.

3. A synoviocyte comprising an artificial chromosome.

4. The synoviocyte of claim 3, wherein the artificial chromosome is an ACes.

5. The synoviocyte of claim 1 that is a fibroblast-like synoviocyte.

6. The synoviocyte of claim 1 that is a mammalian synoviocyte.

7. The synoviocyte of claim 6 that is a primate synoviocyte.

8. The synoviocyte of claim 7 that is a rodent, rabbit, monkey or human synoviocyte.

9. A method for introducing heterologous nucleic acid into a synoviocyte, comprising introducing an artificial chromosome into the synoviocyte.

10. The method of claim 9, wherein the artificial chromosome is an ACes.

11. The method of claim 9, wherein the synoviocyte is a fibroblast-like synoviocyte.

12. A method for introducing a large nucleic acid molecule into a synoviocyte, comprising:
(a) exposing the nucleic acid molecule to a delivery agent; and
(b) contacting the synoviocyte with the nucleic acid molecule.

13. The method of claim 12, wherein the large nucleic acid molecule is an artificial chromosome.

14. The method of claim 13, wherein the artificial chromosome is an ACes.

15. The method of claim 12, wherein the synoviocyte is a fibroblast-like synoviocyte.

16. The method of claim 12, wherein the delivery agent comprises a cationic compound.

17. The method of claim 12, further comprising comprising:
(c) exposing the synoviocyte to a delivery agent, wherein steps (a)-(c) are performed sequentially in any order or simultaneously.

18. The method of claim 17, wherein the synoviocyte is a fibroblast-like synoviocyte.

19. The method of claim 17, wherein the delivery agent comprises a cationic compound.

20. The method of claim 17, wherein the nucleic acid molecule is greater than at least about 0.6 megabase in size.

21. A method for delivering a nucleic acid molecule into a synoviocyte comprising:
(a) contacting the synoviocyte in the absence of the nucleic acid molecule with a delivery agent, and applying ultrasound energy or electrical energy to the synoviocyte, wherein the contacting and applying are performed sequentially or simultaneously; and then
(b) contacting the synoviocyte with the nucleic acid molecule, whereby the nucleic acid molecule is delivered into the synoviocyte.

22. A method for delivering a nucleic acid molecule into a synoviocyte comprising:
(a) contacting the synoviocyte with the nucleic acid molecule and a delivery agent, and applying ultrasound energy or electrical energy to the synoviocyte, wherein the contacting and applying are performed sequentially or simultaneously and whereby the nucleic acid molecule is delivered into the synoviocyte.

23. The method of claim 21, wherein the delivery agent comprises a cationic compound.

24. The method of claim 21, wherein the synoviocyte is a fibroblast-like synoviocyte.

25. The method of claim 21, wherein the energy is ultrasound.

26. A method for modulating a rheumatic disease process in a subject, comprising, introducing a large nucleic acid into the subject; wherein the large nucleic acid comprises nucleic acid that is or that encodes an agent that modulates a rheumatic disease process.

27. The method of claim 26, wherein the nucleic acid is introduced into a site of inflammation in the subject.

28. The method of claim 26, wherein the rheumatic disease process is inflammation.

29. The method of claim 26, wherein the rheumatic disease is rheumatoid arthritis.

30. The method of claim 26, wherein the large nucleic acid is an artificial chromosome.

31. The method of claim 30, wherein the artificial chromosome is an ACes.

32. The method of claim 26, wherein the site of inflammation is in a joint.

33. The method of claim 26, wherein the nucleic acid is introduced into a cell in vitro and then is transferred into the subject.

34. The method of claim 26, wherein the nucleic acid is introduced into a cell in vivo.

35. The method of claim 33, wherein the nucleic acid is introduced into a synoviocyte.

36. The method of claim 33, wherein the synoviocyte is a fibroblast-like synoviocyte.

37. The method of claim 26, wherein the nucleic acid comprises nucleic acid encoding and anti-inflammatory or immunomodulatory molecule.

38. The method of claim 26, wherein the nucleic acid comprises nucleic acid encoding an interleukin-1 receptor antagonist, soluble interleukin-1 receptor, soluble tumor necrosis factor receptor, interferon-β, interleukin-4, interleukin-10, interleukin-13, transforming growth factor β, dominant negative IkappaB-kinase, FasL, Fas-associated death domain protein or CTLA-4 or any combination thereof.

39. A method for identifying candidate agents for the treatment of a connective tissue disease, comprising:
introducing a large nucleic acid molecule comprising nucleic acid that is or encodes a candidate agent into an animal model of the disease; and
determining if the agent ameliorates one or more conditions of the disease in the animal.

40. The method of claim 39, wherein the disease is a rheumatic disease.

41. The method of claim 39, wherein the disease is rheumatoid arthritis.

42. The method of claim 39, wherein the animal is a mammal.

43. The method of claim 39, wherein the animal is a rodent, rabbit, dog, horse or primate.

44. The method of claim 39, wherein the large nucleic acid molecule is an artificial chromosome.

45. The method of claim 39, wherein the artificial chromosome is an ACes.

46. The method of claim 39, wherein the nucleic acid molecule is in a synoviocyte.

47. The method of claim 39, wherein the synoviocyte is a fibroblast-like synoviocyte.

48. The method of claim 39, wherein the nucleic acid is introduced into a joint of the animal.

49. The method of claim 39, wherein the nucleic acid is introduced into a cell in vitro and then is transferred into the animal.

50. The method of claim 39, wherein the nucleic acid is introduced into a cell in vivo.

51. A composition, comprising cells selected for therapeutic treatment of a joint, wherein the cells comprise a large heterologous nucleic acid.

52. The composition of claim 51, wherein the heterologous nucleic acid is an artificial chromosome.

53. The composition of claim 52, wherein the artificial chromosome is an ACes.

54. A composition, comprising cells selected for therapeutic treatment of rheumatoid arthritis, wherein the cells comprise a large heterologous nucleic acid.

55. The composition of claim 54, wherein the heterologous nucleic acid is an artificial chromosome.

56. The composition of claim 55, wherein the artificial chromosome is an ACes.


SEQUENCE LISTING (13 sequences)

SEQ ID NO: 1 - DNA, 24 bp, Artificial Sequence
  Primer
cgtccaggagcgcaccatcttctt

SEQ ID NO: 2 - DNA, 24 bp, Artificial Sequence
  Primer
atcgcgcttctcgttggggtcttt

SEQ ID NO: 3 - DNA, 30 bp, Artificial Sequence
  Primer
aggactgggtggcttccaactcccagacac

SEQ ID NO: 4 - DNA, 30 bp, Artificial Sequence
  Primer
agcttctcattgctgcgcgccaggttcagg

SEQ ID NO: 5 - DNA, 22118 bp, Mus musculus
gaattcccctatccctaatccagattggtggaataacttggtatagatgtttgtgcattaaaaaccctgtaggatcttcactctaggtcactgttcagcactggaacctgaattgtggccctgagtgataggtcctgggacatatgcagttctgcacagacagacagacagacagacagacagacagacagacagacgttacaaacaaacacgttgagccgtgtgccaacacacacacaaacaccactctggccataattattgaggacgttgatttattattctgtgtttgtgagtctgtctgtctgtctgtctgtctgtctgtctgtctatcaaaccaaaagaaaccaaacaattatgcctgcctgcctgcctgcctgcctacacagagaaatgatttcttcaatcaatctaaaacgacctcctaagtttgccttttttctctttctttatctttttcttttttcttttcttcttccttccttccttccttccttccttccttcctttctttctttctttctttctttcttactttctttctttccttcttacatttattcttttcatacatagtttcttagtgtaagcatccctgactgtcttgaagacactttgtaggcctcaatcctgtaagagccttcctctgcttttcaaatgctggcatgaatgttgtacctcactatgaccagcttagtcttcaagtctgagttactggaaaggagttccaagaagactggttatatttttcatttattattgcattttaattaaaatttaatttcaccaaaagaatttagactgaccaattcagagtctgccgtttaaaagcataaggaaaaagtaggagaaaaacgtgaggctgtctgtggatggtcgaggctgctttagggagcctcgtcaccattctgcacttgcaaaccgggccactagaacccggtgaagggagaaaccaaagcgacctggaaacaataggtcacatgaaggccagccacctccatcttgttgtgcgggagttcagttagcagacaagatggctgccatgcacatgttgtctttcagcttggtgaggtcaaagtacaaccgagtcacagaacaaggaagtatacacagtgagttccaggtcagccagagtttacacagagaaaccacatcttgaaaaaaacaaaaaaataaattaaataaatataatttaaaaatttaaaaatagccgggagtgatggcgcatgtctttaatcccagctctcttcaggcagagatgggaggatttctgagtttgaggccagcctggtctgcaaagtgagttccaggacagtcagggctatacagagaaaccctgtcttgaaaactaaactaaattaaactaaactaaactaaaaaaatataaaataaaaattttaaagaattttaaaaaactacagaaatcaaacataagcccacgagatggcaagtaactgcaatcatagcagaaatattatacacacacacacacacagactctgtcataaaatccaatgtgccttcatgatgatcaaatttcgatagtcagtaatactagaagaatcatatgtctgaaaataaaagccagaaccttttctgcttttgttttcttttgccccaagatagggtttctctcagtgtatccctggcatccctgcctggaacttcctttgtaggtttggtagcctcaaactcagagaggtcctctctgcctgcctgcctgcctgcctgcctgcctgcctgcctgcctgcctgcctcacttcttctgccacccacacaaccgagtcgaacctaggatctttatttctttctctttctctcttctttctttctttctttctttctttctttctttctttctttctttctttcttattcaattagttttcaatgtaagtgtgtgtttgtgctctatctgctgcctataggcctgcttgccaggagagggcaacagaacctaggagaaaccaccatgcagctcctgagaataagtgaaaaaacaacaaaaaaaggaaattctaatcacatagaatgtagatatatgccgaggctgtcagagtgctttttaaggcttagtgtaagtaatgaaaattgttgtgtgtcttttatccaaacacagaagagaggtggctcggcctgcatgtctgttgtctgcatgtagaccaggctggccttgaacacattaatctgtctgcctctgcttccctaatgctgcgattaaaggcatgtgccaccactgcccggactgatttcttcttttttttttttttggaaaatacctttctttctttttctctctctctttcttccttccttcctttctttctattctttttttctttcttttttctttttttttttttttttaaaatttgcctaaggttaaaggtgtgctccacaattgcctcagctctgctctaattctctttaaaaaaaaacaaacaaaaaaaaaaccaaaacagtatgtatgtatgtatatttagaagaaatactaatccattaataactcttttttcctaaaattcatgtcattcttgttccacaaagtgagttccaggacttaccagagaaaccctgtgttcaaatttctgtgttcaaggtcaccctggcttacaaagtgagttccaagtccgatagggctacacagaaaaaccatatctcagaaaaaaaaaaagttccaaacacacacacacacacacacacacacacacacacacacacacacacacacacacacagcgcgccgcggcgatgaggggaagtcgtgcctaaaataaatatttttctggccaaagtgaaagcaaatcactatgaagaggtactcctagaaaaaataaatacaaacgggctttttaatcattccagcactgttttaatttaactctgaatttagtcttggaaaagggggcgggtgtgggtgagtgagggcgagcgagcagacgggcgggcgggcgggtgagtggccggcggcggtggcagcgagcaccagaaaacaacaaaccccaagcggtagagtgttttaaaaatgagacctaaatgtggtggaacggaggtcgccgccaccctcctcttccactgcttagatgctcccttccccttactgtgctcccttcccctaactgtgcctaactgtgcctgttccctcaccccgctgattcgccagcgacgtactttgacttcaagaacgattttgcctgttttcaccgctccctgtcatactttcgtttttgggtgcccgagtctagcccgttcgctatgttcgggcgggacgatggggaccgtttgtgccactcgggagaagtggtgggtgggtacgctgctccgtcgtgcgtgcgtgagtgccggaacctgagctcgggagaccctccggagagacagaatgagtgagtgaatgtggcggcgcgtgacggatctgtattggtttgtatggttgatcgagaccattgtcgggcgacacctagtggtgacaagtttcgggaacgctccaggcctctcaggttggtgacacaggagagggaagtgcctgtggtgaggcgaccagggtgacaggaggccgggcaagcaggcgggagcgtctcggagatggtgtcgtgtttaaggacggtctctaacaaggaggtcgtacagggagatggccaaagcagaccgagttgctgtacgcccttttgggaaaaatgctagggttggtggcaacgttactaggtcgaccagaaggcttaagtcctaccccccccccccttttttttttttttcctccagaagccctctcttgtccccgtcaccgggggcaccgtacatctgaggccgagaggacgcgatgggcccggcttccaagccggtgtggctcggccagctggcgcttcgggtctttttttttttttttttttttttttcctccagaagccttgtctgtcgctgtcaccgggggcgctgtacttctgaggccgagaggacgcgatgggccccggcttccaagccggtgtggctcggccagctggagcttcgggtctttttttttttttttttttttttttttctccagaagccttgtctgtcgctgtcaccgggggcgctgtacttctgaggccgagaggacgcgatgggtcggcttccaagccgatgtggcggggccagctggagcttcgggtttttttttttcctccagaagccctctcttgtccccgtcaccgggggcgctgtacttctgaggccgagaggacgtgatgggcccgggttccaggcggatgtcgcccggtcagctggagctttggatctttttttttttttttcctccagaagccctctcttgtccccgtcaccgggggcaccttacatctgagggcgagaggacgtgatgggtccggcttccaagccgatgtggcggggccagctggagcttcgggttttttttttttcctccagaagccctctcttgtccccgtcaccgggggcgctgtacttctgaggccgagaggacgtgatgggcccgggttccaggcggatgtcgcccggtcagctggagctttggatcatttttttttttccctccagaagccctctcttgtccccgtcaccgggggcaccgtacatctgaggccgagaggacacgatgggcctgtcttccaagccgatgtggcccggccagctggagcttcgggtctttttttttttttttcctccagaagccttgtctgtcgctgtcacccggggcgctgtacttctgaggccgagaggacgcgatgggcccggcttccaagccggtgtggctcggccagctggagcttcgggtctttttttttttttttttttttcctccagaaaccttgtctgtcgctgtcacccggggcgcttgtacttctgatgccgagaggacgcgatgggcccgtcttccaggccgatgtggcccggtcagctggagctttggatcttttttttttttttttcctccagaagccctctcttgtccccgtcaccgggggcaccttacatctgaggcctagaggacacgatgggcccgggttccaggccgatgtggcccggtcagctggagctttggatcttttttttttttttcttccagaagccctcttgtccccgtcaccggtggcactgtacatctgaggcggagaggacattatgggcccggcttccaatccgatgtggcccggtcagctggagctttggatcttattttttttttaattttttcttccagaagccctcttgtccctgtcaccggtggcacggtacatctgaggccgagaggacattatgggcccggcttccaggccgatgtggcccggtcagctggagctttggatctttttttttttttttcttttttcctccagaagccctctctgtccctgtcaccgggggccctgtacgtctgaggccgagggaaagctatgggcgcggttttctttcattgacctgtcggtcttatcagttctccgggttgtcagggtcgaccagttgttcctttgaggtccggttcttttcgttatggggtcatttttgggccacctccccaggtatgacttccaggcgtcgttgctcgcctgtcactttcctccctgtctcttttatgcttgtgatcttttctatctgttcctattggacctggagataggtactgacacgctgtcctttccctattaacactaaaggacactataaagagaccctttcgatttaaggctgttttgcttgtccagcctattctttttactggcttgggtctgtcgcggtgcctgaagctgtccccgagccacgcttcctgctttcccgggcttgctgcttgcgtgtgcttgctgtgggcagcttgtgacaactgggcgctgtgactttgctgcgtgtcagacgtttttcccgatttccccgaggtgtcgttgtcacacctgtcccggttggaatggtggagccagctgtggttgagggccaccttatttcggctcactttttttttttttttttctcttggagtcccgaacctccgctcttttctcttcccggtctttcttccacatgcctcccgagtgcatttctttttgttttttttctttttttttttttttttttggggaggtggagagtcccgagtacttcactcctgtctgtggtgtccaagtgttcatgccacgtgcctcccgagtgcacttttttttgtggcagtcgctcgttgtgttctcttgttctgtgtctgcccgtatcagtaactgtcttgccccgcgtgtaagacattcctatctcgcttgtttctcccgattgcgcgtcgttgctcactcttagatcgatgtggtgctccggagttctcttcgggccagggccaagccgcgccaggcgagggacggacattcatggcgaatggcggccgctcttctcgttctgccagcgggccctcgtctctccaccccatccgtctgccggtggtgtgtggaaggcaggggtgcggctctccggcccgacgctgccccgcgcgcacttttctcagtggttcgcgtggtccttgtggatgtgtgaggcgcccggttgtgccctcacgtgtttcactttggtcgtgtctcgcttgaccatgttcccagagtcggtggatgtggccggtggcgttgcatacccttcccgtctggtgtgtgcacgcgctgtttcttgtaagcgtcgaggtgctcctggagcgttccaggtttgtctcctaggtgcctgcttctgagctggtggtggcgctccccattccctggtgtgcctccggtgctccgtctggctgtgtgccttcccgtttgtgtctgagaagcccgtgagaggggggtcgaggagagaaggaggggcaagaccccccttcttcgtcgggtgaggcgcccaccccgcgactagtacgcctgtgcgtagggctggtgctgagcggtcgcggctggggttggaaagtttctcgagagactcattgctttcccgtggggagctttgagaggcctggctttcgggggggaccggttgcagggtctcccctgtccgcggatgctcagaatgcccttggaagagaaccttcctgttgccgcagacccccccgcgcggtcgcccgcgtgttggtcttctggtttccctgtgtgctcgtcgcatgcatcctctctcggtggccggggctcgtcggggttttgggtccgtcccgccctcagtgagaaagtttccttctctagctatcttccggaaagggtgcgggcttcttacggtctcgaggggtctctcccgaatggtcccctggagggctcgccccctgaccgcctcccgcgcgcgcagcgtttgctctctcgtctaccgcggcccgcggcctccccgctccgagttcggggagggatcacgcggggcagagcctgtctgtcgtcctgccgttgctgcggagcatgtggctcggcttgtgtggttggtggctggggagagggctccgtgcacacccccgcgtgcgcgtactttcctcccctcctgagggccgccgtgcggacggggtgtgggtaggcgacggtgggctcccgggtccccacccgtcttcccgtgcctcacccgtgccttccgtcgcgtgcgtccctctcgctcgcgtccacgactttggccgctcccgcgacggcggcctgcgccgcgcgtggtgcgtgctgtgtgcttctcgggctgtgtggttgtgtcgcctcgccccccccttcccgcggcagcgttcccacggctggcgaaatcgcgggagtcctccttcccctcctcggggtcgagagggtccgtgtctggcgttgattgatctcgctctcggggacgggaccgttctgtgggagaacggctgttggccgcgtccggcgcgacgtcggacgtggggacccactgccgctcgggggtcttcgtcggtaggcatcggtgtgtcggcatcggtctctctctcgtgtcggtgtcgcctcctcgggctcccggggggccgtcgtgtttcgggtcggctcggcgctgcaggtgtggtgggactgctcaggggagtggtgcagtgtgattcccgccggttttgcctcgcgtgccctgaccggtccgacgcccgagcggtctctcggtcccttgtgaggacccccttccgggaggggcccgtttcggccgcccttgccgtcgtcgccggccctcgttctgctgtgtcgttcccccctccccgctcgccgcagccggtcttttttcctctctccccccctctcctctgactgacccgtggccgtgctgtcggaccccccgcatgggggcggccgggcacgtacgcgtccgggcggtcaccggggtcttgggggggggccgaggggtaagaaagtcggctcggcgggcgggaggagctgtggtttggagggcgtcccggccccgcggccgtggcggtgtcttgcgcggtcttggagagggctgcgtgcgaggggaaaaggttgccccgcgagggcaaagggaaagaggctagcagtggtcattgtcccgacggtgtggtggtctgttggccgaggtgcgtctggggggctcgtccggccctgtcgtccgtcgggaaggcgcgtgttggggcctgccggagtgccgaggtgggtaccctggcggtgggattaaccccgcgcgcgtgtcccggtgtggcggtgggggctccggtcgatgtctacctccctctccccgaggtctcaggccttctccgcgcgggctctcggccctcccctcgttcctccctctcgcggggttcaagtcgctcgtcgacctcccctcctccgtccttccatctctcgcgcaatggcgccgcccgagttcacggtgggttcgtcctccgcctccgcttctcgccgggggctggccgctgtccggtctctcctgcccgacccccgttggcgtggtcttctctcgccggcttcgcggactcctggcttcgcccggagggtcagggggcttcccggttccccgacgttgcgcctcgctgctgtgtgcttggggggggcccgctgcggcctccgcccgcccgtgagcccctgccgcacccgccggtgtgcggtttcgcgccgcggtcagttgggccctggcgttgtgtcgcgtcgggagcgtgtccgcctcgcggcggctagacgcgggtgtcgccgggctccgacgggtggcctatccagggctcgcccccgccgacccccgcctgcccgtcccggtggtggtcgttggtgtggggagtgaatggtgctaccggtcattccctcccgcgtggtttgactgtctcgccggtgtcgcgcttctctttccgccaacccccacgccaacccaccaccctgctctcccggcccggtgcggtcgacgttccggctctcccgatgccgaggggttcgggatttgtgccggggacggaggggagagcgggtaagagaggtgtcggagagctgtcccggggcgacgctcgggttggctttgccgcgtgcgtgtgctcgcggacgggttttgtcggaccccgacggggtcggtccggccgcatgcactctcccgttccgcgcgagcgcccgcccggctcacccccggtttgtcctcccgcgaggctctccgccgccgccgcctcctcctcctctctcgcgctctctgtcccgcctggtcctgtcccacccccgacgctccgctcgcgcttccttacctggttgatcctgccaggtagcatatgcttgtctcaaagattaagccatgcatgtctaagtacgcacggccggtacagtgaaactgcgaatggctcattaaatcagttatggttcctttggtcgctcgctcctctcctacttggataactgtggtaattctagagctaatacatgccgacgggcgctgaccccccttcccggggggggatgcgtgcatttatcagatcaaaaccaacccggtgagctccctcccggctccggccgggggtcgggcgccggcggcttggtgactctagataacctcgggccgatcgcacgccccccgtggcggcgacgacccattcgaacgtctgccctatcaactttcgatggtagtcgccgtgcctaccatggtgaccacgggtgacggggaatcagggttcgattccggagagggagcctgagaaacggctaccacatccaaggaaggcagcaggcgcgcaaattacccactcccgacccggggaggtagtgacgaaaaataacaatacaggactctttcgaggccctgtaattggaatgagtccactttaaatcctttaacgaggatccattggagggcaagtctggtgccagcagccgcggtaattccagctccaatagcgtatattaaagttgctgcagttaaaaagctcgtagttggatcttgggagcgggcgggcggtccgccgcgaggcgagtcaccgcccgtccccgccccttgcctctcggcgccccctcgatgctcttagctgagtgtcccgcggggcccgaagcgtttactttgaaaaaattagagtgttcaaagcaggcccgagccgcctggataccgcagctaggaataatggaataggaccgcggttctattttgttggttttcggaactgaggccatgattaagagggacggccgggggcattcgtattgcgccgctagaggtgaaattcttggaccggcgcaagacggaccagagcgaaagcatttgccaagaatgttttcattaatcaagaacgaaagtcggaggttcgaagacgatcagataccgtcgtagttccgaccataaacgatgccgactggcgatgcggcggcgttattcccatgacccgccgggcagcttccgggaaaccaaagtctttgggttccggggggagtatggttgcaaagctgaaacttaaaggaattgacggaagggcaccaccaggagtgggcctgcggcttaatttgactcaacacgggaaacctcacccggcccggacacggacaggattgacagattgatagctctttctcgattccgtgggtggtggtgcatggccgttcttagttggtggagcgatttgtctggttaattccgataacgaacgagactctggcatgctaactagttacgcgacccccgagcggtcggcgtcccccaacttcttagagggacaagtggcgttcagccacccgagattgagcaataacaggtctgtgatgcccttagatgtccggggctgcacgcgcgctacactgactggctcagcgtgtgcctaccctgcgccggcaggcgcgggtaacccgttgaaccccattcgtgatggggatcggggattgcaattattccccatgaacgaggaattcccagtaagtgcgggtcataagcttgcgttgattaagtccctgccctttgtacacaccgcccgtcgctactaccgattggatggtttagtgaggccctcggatcggccccgccggggtcggcccacggccctggcggagcgctgagaagacggtcgaacttgactatctagaggaagtaaaagtcgtaacaaggtttccgtaggtgaacctgcggaaggatcattaaacgggagactgtggaggagcggcggcgtggcccgctctccccgtcttgtgtgtgtcctcgccgggaggcgcgtgcgtcccgggtcccgtcgcccgcgtgtggagcgaggtgtctggagtgaggtgagagaaggggtgggtggggtcggtctgggtccgtctgggaccgcctccgatttcccctccccctcccctctccctcgtccggctctgacctcgccaccctaccgcggcggcggctgctcgcgggcgtcttgcctctttcccgtccggctcttccgtgtctacgaggggcggtacgtcgttacgggtttttgacccgtcccgggggcgttcggtcgtcggggcgcgcgctttgctctcccggcacccatccccgccgcggctctggcttttctacgttggctggggcggttgtcgcgtgtggggggatgtgagtgtcgcgtgtgggctcgcccgtcccgatgccacgcttttctggcctcgcgtgtcctccccgctcctgtcccgggtacctagctgtcgcgttccggcgcggaggtttaaggaccccgggggggtcgccctgccgcccccagggtcggggggcggtggggcccgtagggaagtcggtcgttcgggcggctctccctcagactccatgaccctcctccccccgctgccgccgttcccgaggcggcggtcgtgtgggggggtggatgtctggagccccctcgggcgccgtgggggcccgacccgcgccgccggcttgcccgatttccgcgggtcggtcctgtcggtgccggtcgtgggttcccgtgtcgttcccgtgtttttccgctcccgaccctttttttttcctcccccccacacgtgtctcgtttcgttcctgctggccggcctgaggctacccctcggtccatctgttctcctctctctccggggagaggagggcggtggtcgttgggggactgtgccgtcgtcagcacccgtgagttcgctcacacccgaaataccgatacgactcttagcggtggatcactcggctcgtgcgtcgatgaagaacgcagctagctgcgagaattaatgtgaattgcaggacacattgatcatcgacacttcgaacgcacttgcggccccgggttcctcccggggctacgcctgtctgagcgtcggttgacgatcaatcgcgtcacccgctgcggtgggtgctgcgcggctgggagtttgctcgcagggccaaccccccaacccgggtcgggccctccgtctcccgaagttcagacgtgtgggcggttgtcggtgtggcgcgcgcgcccgcgtcgcggagcctggtctcccccgcgcatccgcgctcgcggcttcttcccgctccgccgttcccgccctcgcccgtgcaccccggtcctggcctcgcgtcggcgcctcccggaccgctgcctcaccagtctttctcggtcccgtgccccgtgggaacccaccgcgcccccgtggcgcccgggggtgggcgcgtccgcatctgctctggtcgaggttggcggttgagggtgtgcgtgcgccgaggtggtggtcggtcccctgcggccgcggggttgtcggggtggcggtcgacgagggccggtcggtcgcctgcggtggttgtctgtgtgtgtttgggtcttgcgctgggggaggcggggtcgaccgctcgcggggttggcgcggtcgcccggcgccgcgcaccctccggcttgtgtggagggagagcgagggcgagaacggagagaggtggtatccccggtggcgttgcgagggagggtttggcgtcccgcgtccgtccgtccctccctccctcggtgggcgccttcgcgccgcacgcggccgctaggggcggtcggggcccgtggcccccgtggctcttcttcgtctccgcttctccttcacccgggcggtacccgctccggcgccggcccgcgggacgccgcggcgtccgtgcgccgatgcgagtcacccccgggtgttgcgagttcggggagggagagggcctcgctgacccgttgcgtcccggcttccctgggggggacccggcgtctgtgggctgtgcgtcccgggggttgcgtgtgagtaagatcctccacccccgccgccctcccctcccgccggcctctcggggaccccctgagacggttcgccggctcgtcctcccgtgccgccgggtgccgtctctttcccgcccgcctcctcgctctcttcttcccgcggctgggcgcgtgtcccccctttctgaccgcgacctcagatcagacgtggcgacccgctgaatttaagcatattagtcagcggaggaaaagaaactaaccaggattccctcagtaacggcgagtgaacagggaagagcccagcgccgaatccccgccgcgcgtcgcggcgtgggaaatgtggcgtacggaagacccactccccggcgccgctcgtggggggcccaagtccttctgatcgaggcccagcccgtggacggtgtgaggccggtagcggccccggcgcgccgggctcgggtcttcccggagtcgggttgcttgggaatgcagcccaaagcgggtggtaaactccatctaaggctaaataccggcacgagaccgatagtcaacaagtaccgtaagggaaagttgaaaagaactttgaagagagagttcaagagggcgtgaaaccgttaagaggtaaacgggtggggtccgcgcagtccgcccggaggattcaacccggcggcgcgcgtccggccgtgcccggtggtcccggcggatctttcccgctccccgttcctcccgacccctccacccgcgcgtcgttcccctcttcctccccgcgtccggcgcctccggcggcgggcgcggggggtggtgtggtggtggcgcgcgggcggggccgggggtggggtcggcgggggaccgcccccggccggcgaccggccgccgccgggcgcacttccaccgtggcggtgcgccgcgaccggctccgggacggccgggaaggcccggtggggaaggtggctcggggggggcggcgcgtctcagggcgcgccgaaccacctcaccccgagtgttacagccctccggccgcgctttcgccgaatcccggggccgaggaagccagatacccgtcgccgcgctctccctctccccccgtccgcctcccgggcgggcgtgggggtgggggccgggccgcccctcccacggcgcgaccgctctcccacccccctccgtcgcctctctcggggcccggtggggggcggggcggactgtccccagtgcgccccgggcgtcgtcgcgccgtcgggtcccggggggaccgtcggtcacgcgtctcccgacgaagccgagcgcacggggtcggcggcgatgtcggctacccacccgacccgtcttgaaacacggaccaaggagtctaacgcgtgcgcgagtcaggggctcgtccgaaagccgccgtggcgcaatgaaggtgaagggccccgcccgggggcccgaggtgggatcccgaggcctctccagtccgccgagggcgcaccaccggcccgtctcgcccgccgcgccggggaggtggagcacgagcgtacgcgttaggacccgaaagatggtgaactatgcttgggcagggcgaagccagaggaaactctggtggaggtccgtagcggtcctgacgtgcaaatcggtcgtccgacctgggtataggggcgaaagactaatcgaaccatctagtagctggttccctccgaagtttccctcaggatagctggcgctctcgctcccgacgtacgcagttttatccggtaaagcgaatgattagaggtcttggggccgaaacgatctcaacctattctcaaactttaaatgggtaagaagcccggctcgctggcgtggagccgggcgtggaatgcgagtgcctagtgggccacttttggtaagcagaactggcgctgcgggatgaaccgaacgccgggttaaggcgcccgatgccgacgctcatcagaccccagaaaaggtgttggttgatatagacagcaggacggtggccatggaagtcggaatccgctaaggagtgtgtaacaactcacctgccgaatcaactagccctgaaaatggatggcgctggagcgtcgggcccatacccggccgtcgccgcagtcggaacggaacgggacgggagcggccgcgggtgcgcgtctctcggggtcgggggtgcgtggcgggggcccgtcccccgcctcccctccgcgcgccgggttcgcccccgcggcgtcgggccccgcggagcctacgccgcgacgagtaggagggccgctgcggtgagccttgaagcctagggcgcgggcccgggtggagccgccgcaggtgcagatcttggtggtagtagcaaatattcaaacgagaactttgaaggccgaagtggagaagggttccatgtgaacagcagttgaacatgggtcagtcggtcctgagagatgggcgagtgccgttccgaagggacgggcgatggcctccgttgccctcggccgatcgaaagggagtcgggttcagatccccgaatccggagtggcggagatgggcgccgcgaggccagtgcggtaacgcgaccgatcccggagaagccggcgggaggcctcggggagagttctcttttctttgtgaagggcagggcgccctggaatgggttcgccccgagagaggggcccgtgccttggaaagcgtcgcggttccggcggcgtccggtgagctctcgctggcccttgaaaatccgggggagagggtgtaaatctcgcgccgggccgtacccatatccgcagcaggtctccaaggtgaacagcctctggcatgttggaacaatgtaggtaagggaagtcggcaagccggatccgtaacttcgggataaggattggctctaagggctgggtcggtcgggctggggcgcgaagcggggctgggcgcgcgccgcggctggacgaggcgccgccgccctctcccacgtccggggagaccccccgtcctttccgcccgggcccgccctcccctcttccccgcggggccccgtcgtcccccgcgtcgtcgccacctctcttcccccctccttcttcccgtcggggggcgggtcgggggtcggcgcgcggcgcgggctccggggcggcgggtccaaccccgcgggggttccggagcgggaggaaccagcggtccccggtggggcggggggcccggacactcggggggccggcggcggcggcgactctggacgcgagccgggcccttcccgtggatcgcctcagctgcggcgggcgtcgcggccgctcccggggagcccggcgggtgccggcgcgggtcccctccccgcggggcctcgctccacccccccatcgcctctcccgaggtgcgtggcgggggcgggcgggcgtgtcccgcgcgtgtggggggaacctccgcgtcggtgttcccccgccgggtccgccccccgggccgcggttttccgcgcggcgcccccgcctcggccggcgcctagcagccgacttagaactggtgcggaccaggggaatccgactgtttaattaaaacaaagcatcgcgaaggcccgcggcgggtgttgacgcgatgtgatttctgcccagtgctctgaatgtcaaagtgaagaaattcaatgaagcgcgggtaaacggcgggagtaactatgactctcttaaggtagccaaatgcctcgtcatctaattagtgacgcgcatgaatggatgaacgagattcccactgtccctacctactatccagcgaaaccacagccaagggaacgggcttggcggaatcagcggggaaagaagaccctgttgagcttgactctagtctggcacggtgaagagacatgagaggtgtagaataagtgggaggcccccggcgcccggccccgtcctcgcgtcggggtcggggcacgccggcctcgcgggccgccggtgaaataccactactctcatcgttttttcactgacccggtgaggcgggggggcgagccccgaggggctctcgcttctggcgccaagcgtccgtcccgcgcgtgcgggcgggcgcgacccgctccggggacagtgccaggtggggagtttgactggggcggtacacctgtcaaacggtaacgcaggtgtcctaaggcgagctcagggaggacagaaacctcccgtggagcagaagggcaaaagctcgcttgatcttgattttcagtacgaatacagaccgtgaaagcggggcctcacgatccttctgaccttttgggttttaagcaggaggtgtcagaaaagttaccacagggataactggcttgtggcggccaagcgttcatagcgacgtcgctttttgatccttcgatgtcggctcttcctatcattgtgaagcagaattcaccaagcgttggattgttcacccactaatagggaacgtgagctgggtttagaccgtcgtgagacaggttagttttaccctactgatgatgtgttgttgccatggtaatcctgctcagtacgagaggaaccgcaggttcagacatttggtgtatgtgcttggctgaggagccaatggggcgaagctaccatctgtgggattatgactgaacgcctctaagtcagaatccgcccaagcggaacgatacggcagcgccgaaggagcctcggttggccccggatagccgggtccccgtccgtcccgctcggcggggtccccgcgtcgccccgcggcggcgcggggtctccccccgccgggcgtcgggaccggggtccggtgcggagagccgttcgtcttgggaaacggggtgcggccggaaagggggccgccctctcgcccgtcacgttgaacgcacgttcgtgtggaacctggcgctaaaccattcgtagacgacctgcttctgggtcggggtttcgtacgtagcagagcagctccctcgctgcgatctattgaaagtcagccctcgacacaagggtttgtctctgcgggctttcccgtcgcacgcccgctcgctcgcacgcgaccgtgtcgccgcccgggcgtcacgggggcggtcgcctcggcccccgcgcggttgcccgaacgaccgtgtggtggttgggggggggatcgtcttctcctccgtctcccgaggacggttcgtttctctttccccttccgtcgctctccttgggtgtgggagcctcgtgccgtcgcgaccgcggcctgccgtcgcctgccgccgcagccccttgccctccggccttggccaagccggagggcggaggagggggatcggcggcggcggcgaccgcggcgcggtgacgcacggtgggatccccatcctcggcgcgtccgtcggggacggccggttggaggggcgggaggggtttttcccgtgaacgccgcgttcggcgccaggcctctggcggccgggggggcgctctctccgcccgagcatccccactcccgcccctcctcttcgcgcgccgcggcggcgacgtgcgtacgaggggaggatgtcgcggtgtggaggcggagagggtccggcgcggcgcctcttccattttttcccccccaacttcggaggtcgaccagtactccgggcgacactttgttttttttttttcccccgatgctggaggtcgaccagatgtccgaaagtgtccccccccccccccccccccggcgcggagcggcggggccactctggactcttttttttttttttttttttttttttaaattcctggaacctttaggtcgaccagttgtccgtcttttactccttcatataggtcgaccagtactccgggtggtactttgtctttttctgaaaatcccagaggtcgaccagatatccgaaagtcctctctttccctttactcttccccacagcgattctcttttttttttttttttttggtgtgcctctttttgacttatatacatgtaaatagtgtgtacgtttatatacttataggaggaggtcgaccagtactccgggcgacactttgtttttttttttttttccaccgatgatggaggtcgaccagatgtccgaaagtgtcccgtcccccccctcccccccccgcgacgcggcgggctcactctggactcttttttttttttttttttttttttaaatttctggaaccttaaggtcgaccagttgtccgtctttcactcattcatataggtcgaccggtggtactttgtctttttctgaaaatcgcagaggtcgaccagatgtcagaaagtctggtggtcgataaattatctgatctagatttgtttttctgtttttcagttttgtgttgttttgtgttgttttgtgttgttttgttttgttttgttttgttttgttttgttttgttttgttttgttttgttttgtgttgtgttgtgttgtgttgtgttgggttgggttgggttgggttgggttgggttgggttgggttgggttgggttgtgttgtttggttttgtgttgtttggtgttgttggttttgttttgtttgctgttgttttgtgttttgcgggtcgaacagttgtccctaaccgagtttttttgtacacaaacatgcactttttttaaaataaatttttaaaataaatgcgaaaatcgaccaattatccctttccttctctctcttttttaaaaattttctttgtgtgtgtgtgtgtgtgtgtgtgtgtgtgtgtgcgtgtgtgtgtgtgtgtgcgtgcagcgtgcgcgcgctcgttttataaatacttataataataggtcgccgggtggtggtagcttcccggactccagaggcagaggcaggcagacttctgagttcgaggccagcctggtctacagaggaaccctgtctcgaaaaatgaaaataaatacatacatacatacatacatacatacatacatacatacatacatacatatgaggttgaccagttgtcaatcctttagaattttgtttttaattaatgtgatagagagatagataatagatagatggatagagtgatacaaatataggtttttttttcagtaaatatgaggttgattaaccacttttccctttttaggtttttttttttttcccctgtccatgtggttgctgggatttgaactcaggaccctggcaggtcaactggaaaacgtgttttctatatatataaatagtggtctgtctgctgtttgtttgtttgcttgcttgcttgcttgcttgcttgcttgcttgcttgcttttttttttcttctgagacagtatttctctgtgtaacctggtgccctgaaactcactctgtagaccagcctggcctcaatcgaactcagaaatcctcctgcctcttgtctacctcccaattttggagtaaaggtgtgctacaccactgcctggcattattatcattatcattattaattttattattagacagaacgaaatcaactagttggtcctgtttcgttaattcatttgaaattagttggaccaattagttggctggtttgggaggtttcttttgtttccgatttgggtgtttgtggggctggggatcaggtatctcaacggaatgcatgaaggttaaggtgagatggctcgatttttgtaaagattacttttcttagtctgaggaaaaaataaaataatattgggctacgtttcattgcttcatttctatttctctttctttctttctttctttcagataaggaggtcggccagttcctcctgccttctggaagatgtaggcattgcattgggaaaagcattgtttgagagatgtgctagtgaaccagagagtttggatgtcaagccgtataatgtttattacaatatagaaaagttctaacaaagtgatctttaacttttttttttttttttctccttctacttctacttgttctcactctgccaccaacgcgctttgtacattgaatgtgagctttgttttgcttaacagacatatattttttcttttggttttgcttgacatggtttccctttctatccgtgcagggttcccagacggccttttgagaataaaatgggaggccagaaccaaagtcttttgaataaagcaccacaactctaacctgtttggctgttttccttcccaaggcacagatctttcccagcatggaaaagcatgtagcagttgtaggacacactagacgagagcaccagatctcattgtgggtggttgtgaaccacccaccatgtggttgcctgggatttgaactcaggatcttcagaagacgagtcagggctctaaaccgatgagccatctctccagccctcctacattccttcttaaggcatgaatgatcccagcatgggaagacagtctgccctctttgtggtatatcaccatatactcaataaaataatgaaatgaatgaagtctccacgtatttatttcttcgagctatctaaattctctcacagcacctccccctcccccacactgcctttctccctatgtttgggtggggctgggggaggggtggggtgggggcagggatctgcatgtcttcttgcaggtctgtgaactatttgcgatggcctggttctctgaactgttgagccttgtctatccagaggctgactggctagttttctacctgaagtccctgagtgatgatttccctgtgaattc

SEQ ID NO: 6 - DNA, 175 bp, Mus Musculus
ctcccgcgcggcccccgtgttcgccgttcccgtggcgcggacaatgcggttgtgcgtccacgtgtgcgtgtccgtgcagtgccgttgtggagtgcctcgctctcctcctcctccccggcagcgttcccacggttggggaccaccggtgacctcgccctcttcgggcctggatccg

SEQ ID NO: 7 - DNA, 755 bp, Mus musculus
ggtctggtgggaattgttgacctcgctctcgggtgcggcctttggggaacggcggggtcggtcgtgcccggcgccggacgtgtgtcggggcccacttcccgctcgagggtggcggtggcggcggcgttggtagtctcccgtgttgcgtcttcccgggctcttggggggggtgccgtcgttttcggggccggcgttgcttggcttacgcaggcttggtttgggactgcctcaggagtcgtgggcggtgtgattcccgccggttttgcctcgcgtctgcctgctttgcctcgggtttgcttggttcgtgtctcgggagcggtggtttttttttttttcgggtcccggggagaggggtttttccgggggacgttcccgtcgccccctgccgccggtgggttttcgtttcgggctgtgttcgtttccccttccccgtttcgccgtcggttctccccggtcggtcggccctctccccggtcggtcgcccggccgtgctgccggacccccccttctgggggggatgcccgggcacgcacgcgtccgggcggccactgtggtccgggagctgctcggcaggcgggtgagccagttggaggggcgtcatgcccccgcgggctcccgtggccgacgcggcgtgttctttgggggggcctgtgcgtgcgggaaggctgcgcacgttgtcggtccttgcgagggaaagaggctttttttttttagggggtcgtccttcgtcgtcccgtcggcggtggatccggcct

SEQ ID NO: 8 - DNA, 463 bp, Mus musculus
ggccgaggtgcgtctgcgggttggggctcgtccggccccgtcgtcctccgggaaggcgtttagcgggtaccgtcgccgcgccgaggtgggcgcacgtcggtgagataaccccgagcgtgtttctggttgttggcggcgggggctccggtcgatgtcttcccctccccctctccccgaggccaggtcagcctccgcctgtgggcttcgtcggccgtctccccccccctcacgtccctcgcgagcgagcccgtccgttcgaccttccttccgccttccccccatctttccgcgctccgttggccccggggttttcacggcgccccccacgctcctccgcctctccgcccgtggtttggacgcctggttccggtctccccgccaaaccccggttgggttggtctccggccccggcttgctcttcgggtctcccaacccccggccggaagggttcgggggttccggg

SEQ ID NO: 9 - DNA, 378 bp, Mus musculus
ggattcttcaggattgaaacccaaaccggttcagtttcctttccggctccggccggggggggcggccccgggcggtttggtgagttagataacctcgggccgatcgcacgccccccgtggcggcgacgacccattcgaacgtctgccctatcaactttcgatggtagtcgatgtgcctaccatggtgaccacgggtgacggggaatcagggttcgattccggagagggagcctgagaaacggctaccacatccaaggaaggcagcaggcgcgcaaattacccactcccgacccggggaggtagtgacgaaaaataacaatacaggactctttcgaggccctgtaattggaatgagtccactttaaatcctttaagcag

SEQ ID NO: 10 - DNA, 378 bp, Mus musculus
gatccattggagggcaagtctggtgccagcagccgcggtaattccagctccaatagcgtatattaaagttgctgcagttaaaaagctcgtagttggatcttgggagcgggcgggcggtccgccgcgaggcgagtcaccgcccgtccccgccccttgcctctcggcgccccctcgatgctcttagctgagttgtcccgcggggcccgaagcgtttactttgaaaaaattagagttgtttcaaagcaggcccgagccgcctggataccgccagctaggaaataatggaataggaccgcggttcctattttgtttggttttcggaactgagcccatgattaagggaaacggccgggggcattcccttattgcgccccccta

SEQ ID NO: 11 - DNA, 719 bp, Mus musculus
ggatctttcccgctccccgttcctcccggcccctccacccgcgcgtctccccccttcttttcccctctccggaggggggggaggtgggggcgcgtgggcggggtcgggggtggggtcggcgggggaccgcccccggccggcaaaaggccgccgccgggcgcacttcaaccgtagcggtgcgccgcgaccggctacgagacggctgggaaggcccgacggggaatgtggctcggggggggcggcgcgtctcagggcgcgccgaaccacctcaccccgagtgttacagccctccggccgcgctttcgcggaatcccggggccgaggggaagcccgatacccgtcgccgcgcttttcccctccccccgtccgcctcccgggcgggcgtgggggtgggggccgggccgcccctcccacgcccgtggtttctctctctcccggtctcggccggtttgggggggggagcccggttgggggcggggcggactgtcctcagtgcgccccgggcgtcgtcgcgccgtcgggcccggggggttctctcggtcacgccgcccccgacgaagccgagcgcacggggtcggcggcgatgtcggctacccacccgacccgtcttgaaacacggaccaaggagtctaacgcgtgcgcgagtcaggggctcgcacgaaagccgccgtggcgcaatgaaggtgaagggccccgtccgggggcccgaggtgggatcc

SEQ ID NO: 12 - DNA, 685 bp, Mus musculus
cgaggcctctccagtccgccgagggcgcaccaccggcccgtctcgcccgccgcgtcggggaggtggagcacgagcgtacgcgttaggacccgaaagatggtgaactatgcctgggcagggcgaagccagaggaaactctggtggaggtccgtagcggtcctgacgtgcaaatcggtcgtccgacctgggtataggggcgaaagactaatcgaaccatctagtagctggttccctccgaagtttccctcaggatagctggcgctctcgcaaccttcggaagcagttttatccgggtaaaggcggaatggattaggaggtcttggggccggaaacgatctcaaactatttctcaaactttaaatgggtaaggaagcccggctcgctggcgtggagccgggcgtggaatgcgagtgcctagtgggccacttttggtaagcagaactggcgctgcgggatgaaccgaacgccgggttaaggcgcccgatgccgacgctcatcagaccccagaaaaggtgttggttgatatagacagcaggacggtggccatggaagtcggaatccgctaaggagtgtgtaacaactcacctgccgaatcaactagccctgaaaatggatggcgctggagcgtcgggcccatacccggccgtcgccggcagtcggaacgggacgggacgggagcggccgc

SEQ ID NO: 13 - DNA, 5162 bp, Artificial Sequence
  Chimeric bacterial plasmid
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatcgatatctgcggccgcgtcgacggaattcagtggatccactagtaacggccgccagtgtgctggaattaattcgctgtctgcgagggccagctgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaactgcaggtcgagcatgcatctagggcggccaattccgcccctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccctagagctcgctgatcagcctcgactgtgcctctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgagtgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc